(19) United States
(12) Patent Application Publication
(10) Pub. No.: US 2024/0344055 A1
(43) Pub. Date: Oct. 17, 2024

(54) METHODS AND SYSTEMS FOR MODIFYING THE CRUMBS HOMOLOGUE-1 (CRB1) GENE

(71) Applicants: The Trustees of Columbia University in the City of New York, New York, NY (US); The Broad Institute, Inc., Cambridge, MA (US); President and Fellows of Harvard College, Cambridge, MA (US)

(72) Inventors: Peter M.J. Quinn, New York, NY (US); Bruna Lopes da Costa, New York, NY (US); Stephen H. Tsang, New York, NY (US); David R. Liu, Cambridge, MA (US)

(21) Appl. No.: 18/618,660

(22) Filed: Mar. 27, 2024

Related U.S. Application Data
(63) Continuation-in-part of application No. PCT/US22/77100, filed on Sep. 27, 2022.
(60) Provisional application No. 63/248,808, filed on Sep. 27, 2021.
(60) Provisional application No. 63/498,372, filed on Apr. 26, 2023.

(51) Int. Cl. C12N 15/10; A61K 38/00; C12N 9/12; C12N 9/22; C12N 15/11

(52) U.S. Cl. CPC C12N 15/102; C12N 9/1276; C12N 9/22; C12N 15/111; A61K 38/00; C07K 2319/85; C12N 2310/20; C12Y 207/07049

(57) ABSTRACT

The present disclosure provides systems, methods, and compositions for modifying the crumbs homologue-1 gene. Particularly the present disclosure provides systems, methods, and compositions for prime editing insertion or correction of mutations in the crumbs homologue-1 gene.

### CROSS-REFERENCE TO RELATED APPLICATIONS

This application is a continuation-in-part of International Application No. PCT/US2022/077100, filed Sep. 27, 2022, and claims the benefit of U.S. Provisional Application Nos. 63/248,808, filed Sep. 27, 2021, and U.S. Provisional Application No. 63/498,372, filed Apr. 26, 2023, the contents of each which are herein incorporated by reference in their entirety.

### STATEMENT REGARDING FEDERALLY SPONSORED RESEARCH OR DEVELOPMENT

This invention was made with government support under EY027285, EY018213, OD020351, AG050437, EY028758, EY024698, EY030580, and EY026682 awarded by the National Institutes of Health. The government has certain rights in the invention.

### FIELD

The present invention relates to systems, methods, and compositions for prime-editing modification of the crumbs homologue-1 gene.

### SEQUENCE LISTING STATEMENT

The contents of the electronic sequence listing titled COLUM-40034-502.xml (Size: 77,319 bytes; and Date of Creation: Mar. 27, 2024) is herein incorporated by reference in its entirety.

### BACKGROUND

The Crumbs complex (CRB) is crucial for cell polarity and epithelial tissue function, having an essential role during retinogenesis. Disruption of the CRB complex will interrupt the precise orchestration of spatiotemporal process during retinal development, such as cell fate choice, division, migration, and differentiation. This can cause retinal degeneration leading to impairment of retinal function and thus vision.

Mutations in the Crumbs homologue-1 (CRB1) gene cause progressive and disabling autosomal recessive retinal dystrophies, including Leber congenital amaurosis 8 (LCA8) and retinitis pigmentosa 12 (RP12). Approximately 80,000 patients are affected worldwide, with a prevalence in the United States of 1 in 86,500. CRB1 mutations exhibit high phenotypic variability, with approximately 310 pathogenic variants reported. According to a meta-analysis, CRB1 gene mutations account for 2.7% and 10.1% of autosomal recessive retinitis pigmentosa (RP) and Leber congenital amaurosis (LCA) cases, respectively, as well as an increasing number of juvenile macular dystrophy cases. LCA is a group of severe, infantile-onset retinal dystrophies that constitute more than 5% of all retinal dystrophies and is the most common cause of inherited blindness in childhood. Despite its prevalence and severity, the pathogenesis of CRB1 LCA remains unclear, and there is no treatment available to date.

The mouse and human retina contain three CRB1 isoforms, CRB1-A, CRB1-B, CRB1-C. CRB1-A and CRB1-B have predominately cell-type specific expression making the choice of gene augmentation strategy currently unclear. Due to the complexity of CRB1 cell-type specific retinal isoform diversity, there is a need for compositions and methods that can be used to treat a subject that has or is at risk of developing a disease characterized by Crumbs homologue 1 (CRB1) mutations.

### SUMMARY

Provided herein are systems for modifying the crumbs homologue-1 (CRB1) gene. In some embodiments, the system comprises a Cas protein, or a nucleic acid encoding thereof; a reverse transcriptase, or a nucleic acid encoding thereof; one or more RNA polynucleotides comprising a spacer sequence and an extension sequence comprising a primer binding sequence (PBS) and a reverse transcriptase template (RTT) sequence; or one or more nucleic acids encoding thereof; and optionally, a nicking guide RNA (ngRNA), or a nucleic acid encoding thereof. In some embodiments, the RTT sequence encodes one or more base substitutions, deletions, or additions to modify the CRB1 gene sequence.

In some embodiments, the spacer sequence and the extension sequence are contained within a single RNA polynucleotide.

In some embodiments, the spacer sequence comprises any of SEQ ID NOs: 1, 17, 23-25, and 36-43. In some embodiments, the extension sequence comprises any of SEQ ID NOs: 44-56, the PBS comprises any of SEQ ID NOs: 4-6, 20-22, 30-31. In some embodiments, the RTT sequence comprises any of SEQ ID NOs: 2-3, 12-13, 14, 18-19, 28-29, and 54-57. In some embodiments, the ngRNA comprises any of SEQ ID NOs: 7-11, 15-16, 23-27, and 32-35.

In some embodiments, the spacer sequence comprises SEQ ID NO: 1, the PBS comprises any of SEQ ID NOs: 4-6, and the RTT sequence comprises any of SEQ ID NOs: 2-3 and 12-13. In some embodiments, the ngRNA comprises any of SEQ ID NOs: 7-11.

In some embodiments, the spacer sequence comprises SEQ ID NO: 1, the PBS comprises SEQ ID NO: 4, and the RTT sequence comprises any of SEQ ID NO: 14. In some embodiments, the ngRNA comprises any of SEQ ID NOs: 15-16.

In some embodiments, the spacer sequence comprises SEQ ID NO: 17, the PBS comprises any of SEQ ID NOs: 20-22, and the RTT sequence comprises any of SEQ ID NOs: 18-19 and 28-29. In some embodiments, the ngRNA comprises any of SEQ ID NOs: 23-27.

In some embodiments, the spacer sequence comprises any of SEQ ID NOs: 23-25, the PBS comprises any of SEQ ID NOs: 20, 22, and 30-31, and the RTT sequence comprises any of SEQ ID NOs: 54-57. In some embodiments, the ngRNA comprises any of SEQ ID NOs: 32-35.

In some embodiments, the spacer sequence comprises any of SEQ ID NOs: 36-43 and the extension sequence comprises any of SEQ ID NOs: 44-56.

In some embodiments, the Cas protein is Cas9 or a variant or fragment thereof. In some embodiments, the Cas protein is a Cas9 nickase. In some embodiments, the Cas protein comprises a Cas protein variant configured to target an expanded range of PAM sequences. In some embodiments, the Cas protein comprises a variant of the Streptococcus pyogenes Cas9 selected from xCas9, Cas9-VQR, SpG and SpRY.

In some embodiments, the Cas protein and the reverse transcriptase are contained within a single fusion protein.

In some embodiments, the CRB1 gene is a mutant CRB1 gene comprising one or more disease-causing mutations. In some embodiments, the RTT sequence corrects at least one disease-causing mutation in the CRB1 gene. In some embodiments, the CRB1 gene is a wild-type CRB1 gene. In some embodiments, the RTT sequence inserts at least one mutation in the CRB1 gene.

Provided herein are methods for modifying the crumbs homologue-1 (CRB1) gene comprising contacting a DNA encoding the CRB1 gene with a system as disclosed herein.

In some embodiments, the DNA encoding the CRB1 gene is in a cell. In some embodiments, the cell is a eukaryotic cell. In some embodiments, the cell is a human cell. In some embodiments, the cell is in vitro. In some embodiments, the cell is ex vivo. In some embodiments, the cell is in vivo.

In some embodiments, the contacting comprises introducing the system into the cell. In some embodiments, the introducing into the cell comprises administering to a subject.

Also provided are methods of treating or preventing a disease or disorder in a subject in need thereof. In some embodiments, the methods comprise administering a system as disclosed herein to the subject. In some embodiments, the disease or disorder is caused or mitigated by mutations in CRB1 gene. In some embodiments, the subject has one or more mutations in the CRB1 gene.

In some embodiments, the disease or disorder comprises a retinal dystrophy. In some embodiments, the disease or disorder comprises autosomal recessive retinitis pigmentosa (RP) or Leber congenital amaurosis (LCA).

In some embodiments, the system is configured for delivery to retinal cells. In some embodiments, the system is configured for delivery to photoreceptor cells and or Müller glial cells.

Other aspects and embodiments of the disclosure will be apparent in light of the following detailed description and related figures.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A is a schematic of the retina, showing the canonical crumbs complex involvement in mediating apical polarity and promoting cell adhesion and interaction. FIGS. 1B and 1C show a visual depiction of the correlation between mutations in the CRB1 gene and its affected isoforms.

FIG. 2 is a schematic of the prime editing mechanism. The spacer anneals with its complementary strand of the DNA (1) directing the H840A SpCas9 nickase to nick the PAM-containing strand (black arrow) of the target DNA (2). The primer binding sequence (PBS) then hybridizes with the nicked DNA (3) initiating the elongation of the free 3′ end according to the reverse transcription template (RTT) sequence that carries the intended edit (4). The newly synthesized strand leads to either 3′ or 5′ flap excision. The excision of the 5′ flap is favored, and it leads to the heteroduplex formation (5). The replacement of the original sequence via endogenous DNA mismatch repair mechanism incorporates the desired mutation at the target site (6).

FIG. 3 is a graph of the editing efficiencies of the combination of pegRNA and nicking sgRNA. Prime editing delivered by nucleofection to p.(Gly1103Arg) patient iPSCs. Editing efficiencies calculated by ICE analysis.

FIGS. 4A and 4B show Sanger sequences of bulk iPSCs from two compound heterozygous p.(Cys948Tyr) patients (FIG. 4A) and two homozygous p.(Gly1103Arg) patients (FIG. 4B), before and after prime editing.

FIGS. 5A and 5B show next-generation sequencing (NGS) of bulk cells before and after prime editing for the correction of the p.(Gly1103 Arg) mutation for Patient 2 (FIG. 5A) and Patient 1 (FIG. 5B). Unedited sequences are SEQ ID NOs: 58 and 59, edited sequence is SEQ ID NO: 60, and edited+indels are SEQ ID NOs: 61 and 62.

FIG. 6 is a table of the most prevalent CRB1 mutations.

FIG. 7 is a graph of the editing efficiencies of the indicated combination of pegRNA and nicking sgRNA for the correction of the c.2843G>A CRB1 mutation. Prime editing components were delivered by nucleofection to heterozygous p.(Cys948Tyr) patient iPSCs. Editing efficiencies were calculated by NGS. Positive editing was considered that at or above the 50% threshold determined by NGS from control (unedited patient cells).

FIGS. 8A and 8B shows installation of the CRB1 mutation in control iPSCs and HEK293 cells. FIG. 8A shows prime editing for knock-in of the c.2843G>A, p.(Cys948Tyr) CRB1 mutation in HEK293 cells. FIG. 8B shows prime editing for knock-in of the c.3307G>A, p. (Gly1103Arg) CRB1 mutation into control iPSC lines. Wild-type sequence is SEQ ID NO: 63. c.2843G>A sequence is SEQ ID NO: 64.

FIGS. 9A-9D show dual AAV delivery of split prime editor. FIG. 9A is a schematic of an exemplary design of an AAV split intein system of prime editing using two different vectors for the expression of the prime editor. FIGS. 9B-9D are schematics of PE-C(C-terminal portion) and two versions of PE-N(N-terminal portion) for insertion of the mutation or correction of the mutation, respectively.

FIG. 10 shows prime editing correction of the c.3307G>A, p.(Gly1103Arg) mutation using a split prime editor.

FIG. 11A is a graph of the distribution of CRB1 Pathogenic Alleles From the CRB1 Leiden Open Variation Database. Analysis of 1259 individual pathogenic or likely pathogenic CRB1 alleles in the Leiden Open Variation Database. The most prevalent mutation types include G>A (28.4%), C>T (12.6%), T>C (11.5%) and deletions (12.2%). FIG. 11B is a graph of the editable alleles by variation consequence from the CRB1 Leiden Open Variation Database. Missense, termination, and deletion represent the predominant editable CRB1 variants.

FIG. 12 shows prime editing design for the top ten most prevalent CRB1 mutations. CRB1 sequence fragments to be edited are from top to bottoms SEQ ID NOs: 65-74, respectively. The spacer and 3′ extension sequences can be found in Table 1: c.614 3′ extension is SEQ ID NO: 53; c.2843 3′ extension is SEQ ID NO: 44; c.2688 3′ extension is SEQ ID NO: 48; c.2234 3′ extension is SEQ ID NO: 45; c. 1576 3′ extension is SEQ ID NO: 51; c.3307 3′ extension is SEQ ID NO: 52; c.613_619del 3′ extension is SEQ ID NO: 49; c.2401 3′ extension is SEQ ID NO: 46; c.2290 3′ extension is SEQ ID NO: 47; and c.498_506del 3′ extension is SEQ ID NO: 50.

FIG. 13 shows base editing designs, using a PAM less Cas9, for the correction of p. (Cys948Tyr) and p.(Gly1103Arg) CRB1 mutations. Both designs predict bystanders formation. c.2843G>A sequences shown are SEQ ID NOs: 75 and 76 with spacer sequences at position 4-7 of SEQ ID NOs: 77-80, respectively. c.3307G>A sequences shown are SEQ ID NOs: 81 and 82 with spacer sequences at position 4-7 of SEQ ID NOs: 83-86, respectively.

FIG. 14 shows the timeline of protocol of differentiation from hiPSCs to retinal organoid.

FIGS. 15A-15D show CRB1 LCA retinal organoids. FIGS. 15A and 15B show disruption of cytoskeleton and apical domain in CRB1 LCA retinal organoids. FIGS. 15C and 15D show CRB1 LCA retinal organoids have thinner ONL but appear thicker overall.

FIG. 16 is images showing CRB1 early RP retinal organoids fail to form photoreceptor segments.

FIGS. 17A and 17B show a split-intein prime editing strategy. FIG. 17A is Sanger sequencing of bulk iPSCs from homozygous p.(Gly1103Arg) patient after prime editing using the split-intein PE strategy. FIG. 17B shows AAV8 transduction of retinal organoid with hGRK1 promoter driving GFP expression. Day 290 retinal organoids.

FIGS. 18A and 18B show development of an all-in-one prime editor. FIG. 18A is a schematic of six different exemplary designs tested. In T1-T4 the orientations of the pegRNA, nsgRNA and their respective promoters are changed. In T5 the pegRNA expression is enhanced. In T6 the nsgRNA expression is enhanced. FIG. 18B shows T1-T6 editing efficiencies in performing+1 CTT insertion at the HEK3 locus. Editing efficiency measured by next generation sequencing (NGS).

FIGS. 19A and 19B are graphs of the comparison of prime editing efficiency as an all-in-one plasmid versus separate plasmids in performing several edits across two genomic loci: FANCF loci (FIG. 19A) and HEK3 loci (FIG. 19B). Editing efficiency measured by NGS.

FIGS. 20A and 20B show non-viral delivery of all-in-one PE system using Lipofectamine Stem Transfection Reagent. FIG. 20A shows optimization of the all-in-one PE delivery to iPSCs using 4, 5, or 6 μL lipofectamine and 1.5, 2.25, or 3 μg DNA. FIG. 20B shows all-in-one PE delivery to iPSCs-derived retinal organoids.

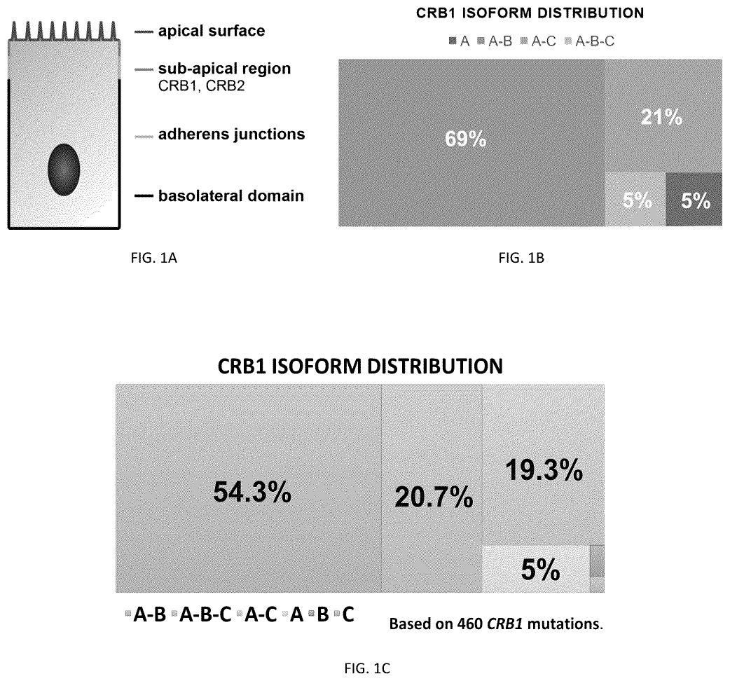

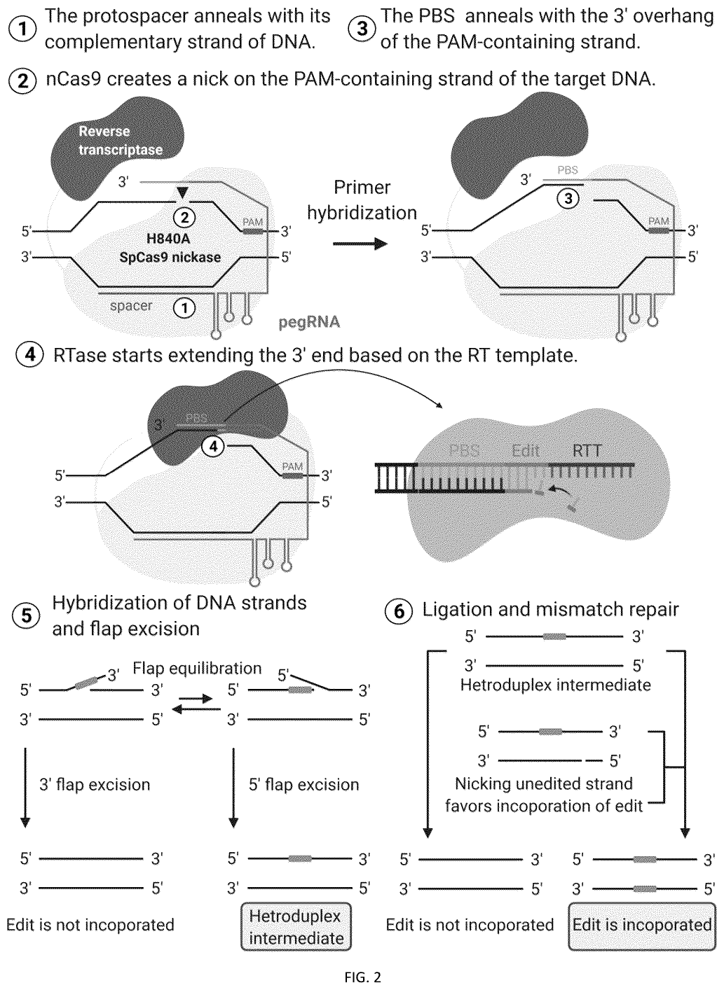

### DETAILED DESCRIPTION

Crumbs (Crb) is a large transmembrane protein initially discovered at the apical membrane of Drosophila epithelial cells. The human CRB1 gene is mapped to chromosome 1q31.3, and contains 12 exons, has 12 identified transcript variants so far, three CRB family members, and over 210 kb genomic DNA. Canonical CRB1 (CRB1-A) is a large transmembrane protein consisting of multiple epidermal growth factor (EGF) and laminin-globular like domains in its extracellular N-terminus. The intracellular C-terminal domain contains a FERM and a conserved glutamic acid-arginine-leucine-isoleucine (ERLI) PDZ binding motives. An alternative transcript of CRB1, CRB1-B, was suggested to have significant extracellular domain overlap with canonical CRB1 while bearing unique 5′ and 3′ domains. In mammals, CRB1 is a member of the Crumbs family together with CRB2 and CRB3.

The disclosed systems, compositions, and methods advance methods to correct or install CRB1 mutations. As shown herein, the disclosed systems, compositions, and methods corrected CRB1 patient mutations and were able to achieve high editing efficiencies.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” and “introducing,” are used interchangeably herein and refer to the placement into a subject by a method or route which results in at least partial localization a desired site. Administration can be by any appropriate route which results in delivery to a desired location in the subject.

The term “contacting” as used herein refers to bring or put in contact, to be in or come into contact. The term “contact” as used herein refers to a state or condition of touching or of immediate or local proximity.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the term “preventing” refers to partially or completely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

As used herein, “treat,” “treating,” and the like means a slowing, stopping, or reversing of progression of a disease or disorder. The term also means a reversing of the progression of such a disease or disorder. As such, “treating” means an application or administration of the methods or devices described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of devices and systems contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, and guinea pigs, and the like. Examples of non-mammals include, but are not limited to, birds, fish, and the like. In one embodiment of the methods herein, the mammal is a human.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (e.g., altered characteristics) when compared to the wild-type gene or gene product. It is noted that naturally-occurring mutants can be isolated; these are identified by the fact that they have altered characteristics when compared to the wild-type gene or gene product.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Prime Editing

Prime editing is a double-strand break (DSB)-independent clustered-regularly interspaced short palindromic repeats (CRISPR)/CRISPR-associated (Cas) system that can ameliorate both transition and transversion mutations in addition to small deletions and insertions. Generally, a prime editing guide RNA (pegRNA) is used in conjunction with a prime editor, e.g., a H840A Streptococcus pyogenes Cas9 (spCas9) nickase linked to an optimized Moloney murine leukemia virus (MMLV) reverse transcriptase (RT).

pegRNAs are similar to standard single-guide RNAs (sgRNAs) but differ due to a 3′ extension sequence comprising a primer binding site (PBS) and an adjacent reverse transcription template (RTT) sequence. The primer binding site hybridizes with the bases upstream of the prime editor generated nick, while the RTT encodes the information of the intended edits and directs reverse transcription. Together, the prime editor and the pegRNA form the prime editing 2 strategy (PE2). The Cas9 nickase is guided to the DNA target site by the pegRNA. After nicking by Cas9, the reverse transcriptase uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Lastly, the prime editor guides resolution of the heteroduplex to favor copying the edit onto the unedited strand, completing the process. Once the prime editor incorporates the edit into one strand, there is a mismatch between the original sequence on one strand and the edited sequence on the other strand. In some embodiments, an additional nicking guide RNA (ngRNA) is used to nick the non-edited strand, directing DNA repair enzymes to use the edited strand as a template to remake the mismatched strand. The prime editor, the pegRNA, and ngRNA form prime editing 3 (PE3) strategies.

Disclosed herein are methods and systems for modifying the sequence of the human crumbs homologue-1 gene. The phrase “modifying” as used herein when referencing a nucleic acid sequence, refers to modifying at least one feature of a nucleic acid sequence. Nucleic acid modifications include, for example, deletion, addition, or insertion of one or more nucleotides to the nucleic acid sequence.

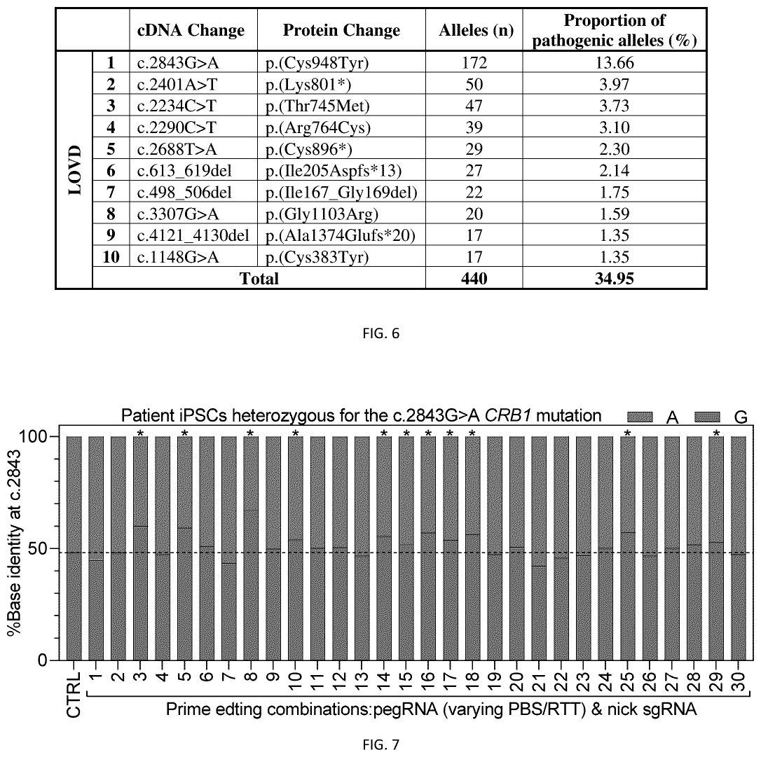

In some embodiments, the methods and systems comprise a sequence-specific nuclease, or a nucleic acid encoding thereof; an RNA-dependent DNA polymerase, or a nucleic acid encoding thereof; one or more RNA polynucleotides comprising a spacer sequence and an extension sequence comprising a primer binding sequence (PBS) and a reverse transcriptase template (RTT) sequence, or one or more nucleic acids encoding thereof; and optionally, a nicking guide RNA (ngRNA), or a nucleic acid encoding thereof. In some embodiments, the RTT sequence encodes one or more nucleotides to modify the crumbs homologue-1 gene. In some embodiments, the RTT sequence is configured to correct any of those mutations found in the table in FIG. 6. In some embodiments, the RTT encodes a wild-type sequence of the crumbs homologue-1 gene.

In some embodiments, systems and methods described herein may be used to insert one or more defects in the crumbs homologue-1 gene. In some embodiments, the RTT sequence encodes a sequence of the crumbs homologue-1 gene having any of those mutations found in the table in FIG. 6 In such cases, the target sequence encodes a wild-type or normal version of the gene, and the disclosed compositions and systems comprise a nucleic acid molecule which encodes a disease-causing version of the gene. For example, the disclosed composition and system may be used to install mutations for disease modeling in cells and organisms.

In some embodiments, the methods comprise contacting the DNA encoding the crumbs homologue-1 gene with the disclosed system. In some embodiments, the methods comprise contacting the DNA encoding the crumbs homologue-1 gene with a Cas9 protein; a reverse transcriptase; one or more RNA polynucleotides comprising a spacer sequence and an extension sequence comprising a primer binding sequence (PBS) and a reverse transcriptase template (RTT) sequence; and optionally, a nicking guide RNA (ngRNA), wherein the RTT sequence encodes one or more nucleotides to correct or insert a mutation in the crumbs homologue-1 gene. In some embodiments, the RTT encodes a wild-type sequence of the crumbs homologue-1 gene. In some embodiments, the RTT sequence encodes a sequence of the crumbs homologue-1 gene having any of those mutations found in the table in FIG. 6

In some embodiments, the DNA encoding the crumbs homologue-1 gene is in a cell. In some embodiments, the cell is a eukaryotic cell. In some embodiments, the cell is a human cell. In some embodiments, the cell is in vitro. In some embodiments, the cell is ex vivo. In some embodiments the cell is in vivo. In some embodiments, the DNA encoding the crumbs homologue-1 gene is genomic DNA. In some embodiments, the crumbs homologue-1 gene is the human crumbs homologue-1 gene.

In some embodiments, contacting the DNA encoding the crumbs homologue-1 gene comprises introducing into the cell: a nucleic acid encoding a Cas9 protein, a nucleic acid encoding a reverse transcriptase; and a nucleic acid encoding one or more RNA polynucleotides comprising a spacer sequence and an extension sequence comprising a primer binding sequence (PBS) and a reverse transcriptase template (RTT) sequence; and, optionally, a nucleic acid encoding a nicking guide RNA (ngRNA). In some embodiments, a single nucleic acid encodes the Cas9 protein and the reverse transcriptase. In some embodiments, a single nucleic acid encodes the spacer sequence, the extension sequence and, optionally, the ngRNA.

In some embodiments, introducing into the cell comprises administering to the subject.

### 1. Sequence-Specific Nuclease

Exemplary sequence-specific nucleases for use in the present invention include, but are not limited to, Cas proteins, Argonaute (Ago) proteins, zinc finger nucleases (ZFNs), and transcription activator-like effector nucleases (TALEN). In some embodiments, the sequence-specific nuclease is a Cas protein.

Cas proteins are described in further detail in, e.g., Haft et al., PLOS Comput. Biol., 1(6): e60 (2005), incorporated herein by reference. The Cas protein may be any Cas endonuclease, or fragment or naturally-occurring or engineered variants thereof. In some embodiments, the Cas endonuclease is a Class 2 Cas endonuclease. In some embodiments, the Cas endonuclease is a Type V Cas endonuclease. In some embodiments, the Cas protein is Cas9, Cas12a, otherwise referred to as Cpf1, or Cas14. In one embodiment, the Cas9 protein is a wild-type Cas9 protein. In some embodiments, the Cas9 protein is a Cas9 variant.

The Cas9 protein can be obtained or derived from any suitable microorganism, and a number of bacteria express Cas9 protein orthologs or variants. In some embodiments, the Cas9 is from Streptococcus pyogenes or Staphylococcus aureus. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases.

In certain embodiments, a Cas nuclease can only cleave a target sequence if an appropriate PAM is present. See, for example Doudna et al., Science, 2014, 346 (6213): 1258096, incorporated herein by reference. A PAM site is a nucleotide sequence in proximity to a target sequence. For example, PAM site may be a DNA sequence immediately following the DNA sequence targeted by the Cas protein. A PAM can be 5′ or 3′ of a target sequence. A PAM can be upstream or downstream of a target sequence. A PAM can be 1, 2, 3, 4, 5, 6, 7, 8, 9, 10 or more nucleotides in length. In certain embodiments, a PAM is between 2-6 nucleotides in length. Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, and NGGNG, where “N” is any nucleotide.

In some embodiments, the Cas protein comprises a Cas variant configured to target an expanded or altered range of PAM sequences which may facilitate essentially PAMless cleavage. In some embodiments, the Cas protein comprises a variant of the Streptococcus pyogenes Cas9 enzyme selected from xCas9, Cas9-VQR, SpG and SpRY. See, for example, Walton et al., Science. 2020 Apr. 17; 368 (6488): 290-296, Hu, et al., Nature 2018; 556 (57-63), Kleinstiver et al., Nature 2015; 523 (7561): 481-5, Hu et al., Mol Plant 2016; 9, 43-945, incorporated herein by reference in their entirety.

In some embodiments, the Cas protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see, e.g., U.S. Patent Application Publication 2017/0051312, incorporated herein by reference) and include, for example, Streptococcus pyogenes with point mutations at D10 or H840.

### 2. RNA-Dependent DNA Polymerase

RNA-dependent DNA polymerases (e.g., reverse transcriptases) synthesize complementary DNA using RNA as a template. Any RNA-dependent DNA polymerase, or variant or truncation thereof or enzyme having RNA-dependent DNA polymerase activity can be utilized in the systems and methods herein. Exemplary RNA-dependent DNA polymerases include retroviral reverse transcriptases, retrotransposon reverse transcriptases, bacterial reverse transcriptases, Tth DNA polymerase, Taq DNA polymerase, and Tma DNA polymerase. In some embodiments, the RNA-dependent DNA polymerases is a moloney murine leukemia virus (MMLV) reverse transcriptase

In some embodiments, the reverse transcriptase and the Cas protein comprise a fusion protein. The reverse transcriptase can be fused to the Cas protein in any orientation and may be separated from the Cas protein with an amino acid linker.

In some embodiments, the prime editor, fusion protein comprising the reverse transcriptase and the Cas protein, is provided as a split-prime editor (e.g., a prime editor can in some cases be delivered as a split-prime editor, or a nucleic acid(s) encoding a split-prime editor) such that two separate proteins together form a functional prime editor. In some such cases the sequences that encode the two parts of the split-prime editor protein are present on the same vector. In some cases, they are present on separate vectors, e.g., as part of a vector system that encodes the prime editor, the gRNA(s), and systems thereof.

Exemplary methods and linkers include split-intein protein trans-splicing known in the art, e.g., for reconstituting the Cas9 protein. A split intein is any intein in which the N-terminal domain of the intein and the C-terminal domain of the intein are not directly linked via a peptide bond. Natural split inteins have been identified in cyanobacteria and archaea, but split inteins can also be created artificially by separating an intein's sequence into two pieces. For example, in some embodiments, a first nucleic acid may encode a first protein having the N-terminal portion of the prime editor fused to one half of a split intein and a second nucleic acid may encode a second protein having the C-terminal portion of the prime editor and the other half of the split intein.

3. pegRNA

The systems and methods disclosed herein include a spacer sequence and an extension sequence comprising a primer binding sequence (PBS) and a reverse transcriptase template (RTT) sequence, or one or more nucleic acids encoding thereof. In some embodiments, each of the spacer sequence, PBS, and RTT sequence are provided as a single prime editing guide RNA (pegRNA), or a nucleic acid encoding thereof. The spacer sequence directs the nuclease to bind to a DNA molecule having complementarity with the pegRNA, the PBS hybridizes with the bases upstream of the nuclease generated nick, and the RTT encodes the information of the intended edits and directs reverse transcription.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization.

The spacer sequence and the extension sequence may be selected from any of the sequences disclosed herein or fragments thereof which lack one or more nucleotides from the 5′ and/or 3′ end. In some embodiments, the spacer sequence or the extension sequence comprises one, two, three, four, five, six, seven, eight, nine, or ten substitutions as compared to the sequences disclosed herein. In some embodiments, the spacer sequence or the extension sequence comprises one, two, three, four, five, six, seven, eight, nine, or ten additional nucleotides on the 5′ and/or 3′ end as compared to the sequences disclosed herein. The spacer sequence and the extension sequence may be optimized by the described additions or substitutions for use in a variety of cell types and methods.

The pegRNAs may comprise additional structural elements or sequences including a gRNA scaffold responsible for Cas9 binding, a transcription termination sequence that the 3′ end of the molecule, and mutations or structural motifs that increase editing efficiency or enhance RNA stability or prevent RNA degradation. For example, the pegRNA may further comprise: a triple helix forming sequence (e.g., triple helix terminators from a long non-coding RNAs (lncRNAs), e.g., metastasis-associated lung adenocarcinoma transcript 1 (MALATI)); a tRNA-like sequence; a pseudoknot (e.g., a modified prequeosine₁-1 riboswitch aptamer, (evopreQ₁) or the frameshifting pseudoknot from Moloney murine leukemia virus (MMLV)); and silent mutations near the intended edit (e.g., less than 10 bp away). See, for example, Nelson, et al. Nat Biotechnol. 2022 March; 40 (3): 402-410, Chen, et al., Cell. 2021 Oct. 28; 184 (22): 5635-5652.e29, International Patent Publication No. WO2022067130, each of which is incorporated herein by reference in its entirety.

The additional structural elements or sequences may be present at any location in the pegRNA which does not interfere with the function of the spacer sequence, primer binding sequence (PBS), and a reverse transcriptase template (RTT) sequence. In some embodiments, the additional structural elements or sequences are at the 3′ end of the pegRNA.

4. Nicking Guide RNA (ngRNA)

In some embodiments, the systems and methods comprise a nicking guide RNA (ngRNA) that complexes with the sequence-specific nuclease and introduces a nick in the non-edited DNA stand. In certain embodiments, the nick induced by using the ngRNA is on the opposite strand as the initial nick. In certain embodiments, the nick induced by using the ngRNA is on the same strand as the initial nick. Thus, the ngRNA sequence may target the same or different strand as the spacer sequence.

The ngRNA may be selected from any of the sequences disclosed herein or fragments thereof which lack one or more nucleotides from the 5′ and/or 3′ end. In some embodiments, the ngRNA comprises one, two, three, four, five, six, seven, eight, nine, or ten substitutions as compared to the sequences disclosed herein. In some embodiments, the ngRNA comprises one, two, three, four, five, six, seven, eight, nine, or ten additional nucleotides on the 5′ and/or 3′ end as compared to the sequences disclosed herein.

The systems and methods may further include an engineered DNA mismatch repair (MMR)-inhibitor (e.g., protein or silencing RNA), or a nucleic acid encoding thereof. See Chen, et al., Cell. 2021 Oct. 28; 184 (22): 5635-5652.e29, incorporated herein by reference.

Nucleic acids of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EFla (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. In some embodiments, the promoter is specific to retinal cells. In some embodiments, the promoter directs expression in rod and/or cone photoreceptor cells.

Additionally, the vector may contain, for example, some or all of the following: a selectable marker gene for selection of stable or transient transfectants in host cells; transcription termination and RNA processing signals; 5′- and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRPI genes of S. cerevisiae.

When introduced into a cell, the vectors may be maintained as an autonomously replicating sequence or extrachromosomal element or may be integrated into host DNA.

Thus, the disclosure further provides for cells comprising a system for modifying the crumbs homologue-1 gene, or one or more nucleic acids or vectors encoding thereof, as disclosed herein.

Viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells, tissues and/or a subject. Viral vectors include, for example, retroviral, lentiviral, adenoviral, adeno-associated, baculoviral, and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant baculoviruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7 (1): 33-40; and Walther W. and Stein U., 2000 Drugs, 60 (2): 249-71, incorporated herein by reference.

Vectors according to the present disclosure can be transformed, transfected, or otherwise introduced into a wide variety of host cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459 (1-2): 70-83), incorporated herein by reference.

As such, the disclosure provides an isolated cell comprising the vector(s) or nucleic acid(s) disclosed herein. Preferred cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14:810-817 (1993); Lucklow, Curr. Opin. Biotechnol., 4:564-572 (1993); and Lucklow et al., J. Virol., 67:4566-4579 (1993), incorporated herein by reference. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines. Methods for selecting suitable mammalian cells and methods for transformation, culture, amplification, screening, and purification of cells are known in the art.

In some embodiments, the cell is a eukaryotic cell. In some embodiments, the cell is a mammalian cell. In some embodiments, the cell is a human cell. In some embodiments, the cell is in vitro. In some embodiments, the cell is ex vivo. In some embodiments, the cell is in vivo and delivery to the cell comprises administration to a subject.

### Methods of Treating a Disease or Disorder

Also disclosed herein are methods for treating or preventing a disease or disorder in a subject caused or mitigated by mutations in the crumbs homologue-1 (CRB1) gene. In some embodiments, the subject has a mutation in in the CRB1 gene. In some embodiments, the subject has a CRB1 gene with any of the mutations shown in FIG. 6.

The methods comprise administering to a subject: a sequence-specific nuclease, or a nucleic acid encoding thereof; an RNA-dependent DNA polymerase, or a nucleic acid encoding thereof; one or more RNA polynucleotides comprising a spacer sequence and an extension sequence comprising a primer binding sequence (PBS) and a reverse transcriptase template (RTT) sequence, or one or more nucleic acids encoding thereof; and optionally, a nicking guide RNA (ngRNA), or a nucleic acid encoding thereof. The systems and components disclosed herein are applicable to the methods for treatment and prevention of a disease or disorder.

In some embodiments, the disease or disorder is an ocular disease or disorder. In some embodiments, the disease or disorder is a retinal disease or disorder. In some embodiments, the disease or disorder comprises retinal degeneration, blindness or severe visual impairment, night blindness, loss of peripheral vision, progressive photoreceptor degeneration, nonrecordable electroretinogram (ERG), nystagmus, hypermetropia, sluggish or absent pupillary responses and oculodigital reflexes, macular dystrophy, foveal schisis, or a combination thereof.

A wide range of retinal dystrophies can be caused by mutations in the CRB1 gene. In some embodiments, disease or disorder characterized by CRB1 mutations includes, but is not limited to, autosomal recessive retinitis pigmentosa (RP) and Leber congenital amaurosis (LCA).

In some embodiments, the systems or components thereof are configured for delivery to retinal cells. In some embodiments, the system is configured for delivery to rod and cone photoreceptor cells and/or Müller glial cells. For example, in some embodiments, the nucleic acids encoding the components may comprise a retinal cell (e.g., rod and/or cone photoreceptor cell or Müller glial cell) promoter which directs expression of the components in the retinal cells. In some embodiments, mini promoters, minimal promoter element(s) designed for expression in specific types (e.g., rod and/or cone photoreceptor cell or Müller glial cell) are used.

Suitable retinal, rod, and/or cone photoreceptor cell promoters include, but are not limited to: 770En_454P (hGRM6), a human GRM6 gene-derived, short promoter; promoters based on the 2.1-kb human L-opsin promoter (pR2.1); promoter derived from the rhodopsin kinase (RK) gene; promoter derived from the rhodopsin gene; a promoter derived from the Nrl gene; murine rhodopsin promoter (mOP); G-protein-coupled receptor protein kinase 1 (GRK1) promoter; retinol-binding protein 3, interstitial (RBP3) promoter; RPE65 promoter; human inter-photoreceptor retinoid binding protein/retinol-binding protein 3 (IRBP) promoter; and retinaldehyde binding protein 1 (RLBP1) promoter. Suitable Müller glial cell promoters include but, are not limited to: RLBP1 (Retinaldehyde Binding Protein 1), GFAP (Glial fibrillary acidic protein), GfaABCID (a truncated GFAP promoter), and synthetic promoters ProB2 and PROC17. Suitable photoreceptor cell promoters include, but are not limited to: interphotoreceptor retinoid-binding protein (IRBP), cone arrestin (CAR), rhodopsin (RHO), PR1.7 (a truncated version of version of the L-opsin promoter), synthetic promoters: ProA1, ProA6, ProC1, ProA14, and ProA36, and G protein-coupled receptor kinase 1 (GRK1).

Additionally, or alternatively, the systems or components are configured for administration to the eye and/or retina, rather than systemic administration.

Administration may be through any suitable mode of administration, including but not limited to: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, topical, and inhalation. In some embodiments, the systems or components are delivered to the tissue(s) of interest. Such delivery may be either via a single dose, or multiple doses.

In some embodiments, an effective amount of the components of the systems, methods or compositions as described can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful modification of the CRB1 gene is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

### EXAMPLES

The following are examples of the present invention and are not to be construed as limiting.

### Materials and Methods

Plasmid Constructs and Cloning. The following plasmids were used: PEmax NGG and NGA (Chen et al., 2021 Cell), pU6-ePegRNA-GG-acceptor (Nelson et al., 2022 Nat Biotechnol) and pU6-spacer-acceptor (Tsai et al., 2022 Methods Mol Biol). The pegRNA was cloned into the pU6-ePegRNA-GG-acceptor and nicking guide RNA (ngRNA) was cloned into the pU6-spacer-acceptor, both using Bsal Golden Gate assembly (NEB), as described previously (Anzalone et al., 2019. Nature; Nelson et al., 2022 Nat Biotechnol; Tsai et al., 2022 Methods Mol Biol). All oligos were ordered from IDT with relevant overhangs for above described cloning methodology. A “G” was added to all spacer sequences that did not start with a G, for high expression the human U6 promoter prefers a “G” at transcription start.

HEK293 Transfection. To test editing efficiency of the prime editing, the plasmids were transfected into HEK293 cells. The HEK293 cells were seeded one day before at 50000 cells/well in 24 well plates. On the next day, the medium was refreshed with 500 ul complete medium. For the transfection, the plasmid constitution is 1050 ng: 393.75 ng: 78.75 ng of CMV-PEmax NGG or CMV-PEmax NGA: U6-pegRNA with append: U6-ngRNA. Lipofectamine 2000 (Thermo Fisher) was mixed at 1:1 mass ratio with plasmid DNA. The cells were then collected after 72 hours post transfection for DNA extraction and analysis.

iPSC Transfection. To test prime editing efficiency in CRB1 patient or control iPSC lines the plasmids were nucleofected into cells using the Lonza P3 Primary Cell Nucleofector Kit and the Amaxa 4D-Nucleofector (Lonza Biosciences). Successful editing was observed with both the DS-150 and CA-137 nucleofection programs. iPSCs were pre-treated for 2 hours with mTeSR plus and rock inhibitor or Clone R2 (Stem Cell Technologies). Cells were lifted using either ReLeSR (Stem Cell Technologies) or TrypLE (Gibco) and counted. 2×10⁵ cells were used and nucleofected with plasmid at the following ratio: 700 ng: 262.5 ng: 52.5 ng of CMV-PEmax NGG or CMV-PEmax NGA: U6-pegRNA with append: U6-ngRNA. Post nucleofection cells were resuspended in 150 ul of mTeSR plus and rock inhibitor or Clone R2 and seeded in matrigel-covered wells in 96 well plate. Medium was refreshed at 48 h post nucleofection. The cells were then collected after 72 hours post transfection for DNA extraction and analysis.

DNA extraction. The cells were detached from the wells by trypsin for HEK293 and ReLeSR (Stem Cell Technologies) for iPSCs. The cells from each well were washed with DPBS (without Ca2+ and Mg2+) and re-suspended with 50 ul DPBS. The cells were then incubated at 95° C. for 20 mins. Subsequently, after cooling, 4 ul of 20 mg/ml Proteinase K (Promega) was added to each sample. The samples were then incubated at 56° C. for one hour followed by a 30 min incubation at 95° C. to stop the proteinase K digestion. This crude extract of DNA is then ready for PCR purpose.

Analysis of Prime Editing Efficiency. For the determination of editing efficiency, the relevant CRB1 locus was amplified using primers with Illumina adaptors. The amplicon was submitted to Genewiz for the Amplicon EZ service. The analysis of the sequencing data was determined using CRISPResso2 (crispresso.pinellolab.partners.org/submission). The reads of the amplicons less than 0.1% of the total frequency were excluded for analysis. In some cases, the CRB1 locus was amplified with primers and editing efficiency was evaluated using ICE analysis (Synthego).

### Correcting Human CRB1 c.2843G>A Pathogenic Mutation, NGA PAM

| | | SEQ ID | | SEQ ID | | SEQ ID |
|---|---|---|---|---|---|---|
| pegRNA_name | protospacer | NO | RT_temp | NO | PBS | NO |
| NGA_PBS11_RTT16 | TATTATCACC | 1 | ATTTGCAAT | 2 | GAGAG | 4 |
| | TTCTCTCATT | | ACCTAAT | | AAGGT | |
| | | | | | G | |
| NGA_PBS13_RTT16 | TATTATCACC | 1 | ATTTGCAAT | 2 | GAGAG | 5 |
| | TTCTCTCATT | | ACCTAAT | | AAGGT | |
| | | | | | GAT | |
| NGA_PBS15_RTT16 | TATTATCACC | 1 | ATTTGCAAT | 2 | GAGAG | 6 |
| | TTCTCTCATT | | ACCTAAT | | AAGGT | |
| | | | | | GATAA | |
| NGA_PBS11_RTT26 | TATTATCACC | 1 | TAAAAACA | 3 | GAGAG | 4 |
| | TTCTCTCATT | | GCATTTGCA | | AAGGT | |
| | | | ATACCTAAT | | G | |
| NGA_PBS13_RTT26 | TATTATCACC | 1 | TAAAAACA | 3 | GAGAG | 5 |
| | TTCTCTCATT | | GCATTTGCA | | AAGGT | |
| | | | ATACCTAAT | | GAT | |
| NGA_PBS15_RTT26 | TATTATCACC | 1 | TAAAAACA | 3 | GAGAG | 6 |
| | TTCTCTCATT | | GCATTTGCA | | AAGGT | |
| | | | ATACCTAAT | | GATAA | |

| | | SEQ ID |
|---|---|---|
| Nicking sgRNA name | protospacer | NO |
| 2843G > A nsgRNA 1 | CATTGCTTCTGAATAATATT | 7 |
| 2843G > A nsgRNA 2 | GGTAATATTCCCATTGCTTC | 8 |
| 2843G > A nsgRNA 3 | GAAACCAAATGTGATATTGG | 9 |
| 2843G > A nsgRNA 4 | CCTTGTTCTGAAACCAAATG | 10 |
| 2843G > A nsgRNA 5 | ATTACCGTTATTAATAGTAA | 11 |

Correcting Human CRB1 c.2843G>A Pathogenic Mutation with Silent Mutation, NGA PAM

| | | SEQ ID | | SEQ ID | | SEQ ID |
|---|---|---|---|---|---|---|
| pegRNA_name | protospacer | NO | RT_temp | NO | PBS | NO |
| SM-948- | TATTATCACC | 1 | ATTTGCgATg | 12 | GAGAGAA | 4 |
| PBS11_RTT16 | TTCTCTCATT | | cCTAAT | | GGTG | |
| SM-948- | TATTATCACC | 1 | ATTTGCgATg | 12 | GAGAGAA | 5 |
| PBS13_RTT16 | TTCTCTCATT | | cCTAAT | | GGTGAT | |
| SM-948- | TATTATCACC | 1 | ATTTGCgATg | 12 | GAGAGAA | 6 |
| PBS15_RTT16 | TTCTCTCATT | | cCTAAT | | GGTGATAA | |
| SM-948- | TATTATCACC | 1 | TAAAAACAG | 13 | GAGAGAA | 4 |
| PBS11_RTT26 | TTCTCTCATT | | CATTTGCgA | | GGTG | |
| | | | TgcCTAAT | | | |
| SM-948- | TATTATCACC | 1 | TAAAAACAG | 13 | GAGAGAA | 5 |
| PBS13_RTT26 | TTCTCTCATT | | CATTTGCgA | | GGTGAT | |
| | | | TgcCTAAT | | | |
| SM-948- | TATTATCACC | 1 | TAAAAACAG | 13 | GAGAGAA | 6 |
| PBS15_RTT26 | TTCTCTCATT | | CATTTGCgA | | GGTGATAA | |
| | | | TgcCTAAT | | | |

### Installing Human CRB1 c.2843G>A Pathogenic Mutation, NGG PAM

| | | SEQ ID | | SEQ ID | | SEQ ID |
|---|---|---|---|---|---|---|
| pegRNA_name | protospacer | NO | RT_temp | NO | PBS | NO |
| 2843G > A | TATTATC | 1 | TAAAAACAG | 14 | GAGAG | 4 |
| Knockin Proto 1 NGG | ACCTTCT | | CATTTGCAA | | AAGGTG | |
| PBS11_RTT26 | CTCATT | | TATCTAAT | | | |

| | | SEQ |
|---|---|---|
| | | ID |
| Nicking sgRNA name | protospacer | NO |
| 2843G > A Knockin- | GTGTGATATTGGTGAGTTCTC | 15 |
| Nick1 | | |
| 2843G > A Knockin- | GTCTGAAACCAAATGTGATAT | 16 |
| Nick2 | | |

### Correcting Human CRB1 c.3307G>A Pathogenic Mutation, NGG PAM

| | | SEQ ID | | SEQ ID | | SEQ ID |
|---|---|---|---|---|---|---|
| pegRNA_name | protospacer | NO | RT_temp | NO | PBS | NO |
| hCRB1- | CTAAGTACA | 17 | AGATAAATG | 18 | ATTTCT | 20 |
| 3307G_Proto_1_ | ATAGAAATC | | CCTCCG | | ATTGT | |
| NGG_PBS11_ | AG | | | | | |
| RTT15 | | | | | | |
| hCRB1- | CTAAGTACA | 17 | AGATAAATG | 18 | ATTTCT | 21 |
| 3307G_Proto_1_ | ATAGAAATC | | CCTCCG | | ATTGTA | |
| NGG_PBS13_ | AG | | | | C | |
| RTT15 | | | | | | |
| hCRB1- | CTAAGTACA | 17 | AGATAAATG | 18 | ATTTCT | 22 |
| 3307G_Proto_1_ | ATAGAAATC | | CCTCCG | | ATTGTA | |
| NGG_PBS15_ | AG | | | | CTT | |
| RTT15 | | | | | | |
| hCRB1- | CTAAGTACA | 17 | AAAGTAAGA | 19 | ATTTCT | 20 |
| 3307G_Proto_1_ | ATAGAAATC | | GAGATAAAT | | ATTGT | |
| NGG_PBS11 | AG | | GCCTCCG | | | |
| RTT25 | | | | | | |
| hCRB1- | CTAAGTACA | 17 | AAAGTAAGA | 19 | ATTTCT | 21 |
| | ATAGAAATC | | GAGATAAAT | | ATTGTA | |
| 3307G_Proto_1_ | AG | | GCCTCCG | | C | |
| NGG_PBS13 | | | | | | |
| RTT25 | | | | | | |
| hCRB1- | CTAAGTACA | 17 | AAAGTAAGA | 19 | ATTTCT | 22 |
| 3307G_Proto_1_ | ATAGAAATC | | GAGATAAAT | | ATTGTA | |
| NGG_PBS15_ | AG | | GCCTCCG | | CTT | |
| RTT25 | | | | | | |

| | | SEQ |
|---|---|---|
| Nicking sgRNA name | protospacer | ID NO |
| 3307G > A nsgRNA 1 | TTGAGAAATTGCTCTTCCTG | 23 |
| 3307G > A nsgRNA 2 | ACAGCCAGTGACCACTGAAT | 24 |
| 3307G > A nsgRNA 3 | TCCTCCATGCAAACAGGGGT | 25 |
| 3307G > A nsgRNA 4 | TATTATCCTTCAAAAAGTTG | 26 |
| 3307G > A nsgRNA 5 | TTGTACTTAGACACCCTTGC | 27 |

Correcting CRB1 c.3307G>A Pathogenic Mutation with Silent Mutations, NGG PAM

| | | SEQ ID | | SEQ ID | | SEQ ID |
|---|---|---|---|---|---|---|
| pegRNA_name | protospacer | NO | RT_temp | NO | PBS | NO |
| SM-1103- | CTAAGTA | 17 | AGATAAATt | 28 | ATTTCTAT | 20 |
| RTT15_PBS11 | CAATAGA | | CCgCca | | TGT | |
| | AATCAG | | | | | |
| SM-1103- | CTAAGTA | 17 | AGATAAATt | 28 | ATTTCTAT | 21 |
| RTT15_PBS13 | CAATAGA | | CCgCca | | TGTAC | |
| | AATCAG | | | | | |
| SM-1103- | CTAAGTA | 17 | AGATAAATt | 28 | ATTTCTAT | 22 |
| RTT15_PBS15 | CAATAGA | | CCgCca | | TGTACTT | |
| | AATCAG | | | | | |
| SM-1103- | CTAAGTA | 17 | AAAGTAAG | 29 | ATTTCTAT | 20 |
| RTT25_PBS11 | CAATAGA | | AGAGATAA | | TGT | |
| | AATCAG | | ATtCCgCca | | | |
| SM-1103- | CTAAGTA | 17 | AAAGTAAG | 29 | ATTTCTAT | 21 |
| RTT25_PBS13 | CAATAGA | | AGAGATAA | | TGTAC | |
| | AATCAG | | ATtCCgCca | | | |
| SM-1103- | CTAAGTA | 17 | AAAGTAAG | 29 | ATTTCTAT | 22 |
| RTT25_PBS15 | CAATAGA | | AGAGATAA | | TGTACTT | |
| | AATCAG | | ATtCCgCca | | | |

### Installing Human CRB1 c.3307G>A Pathogenic Mutation, NGG PAM

| | | SEQ ID | | SEQ ID | | SEQ ID |
|---|---|---|---|---|---|---|
| pegRNA_name | protospacer | NO | RT_temp | NO | PBS | NO |
| 3307G > A Knockin | CTAAGTACA | 23 | AAAGTAAG | 54 | ATTTC | 22 |
| NGG_RTT25_PBS15 | ATAGAAATC | | AGAGATAA | | TATTG | |
| | GG | | ATGCCTCTG | | TACTT | |
| 3307G > A Knockin | CTAAGTACA | 23 | AGATAAAT | 55 | ATTTC | 20 |
| NGG_RTT15_PBS11 | ATAGAAATC | | GCCTCTG | | TATTG | |
| | GG | | | | T | |
| 3307G > A Knockin- | TGTCTAAGTA | 24 | TAAATGCCT | 56 | TCTAT | 30 |
| PBS12-RTT15- | CAATAGAAA | | CtGATT | | TGTAC | |
| Design2 | T | | | | TT | |
| 3307G > A Knockin- | GTAAGAGAG | 25 | CAATAGAA | 57 | GCATT | 31 |
| PBS12-RTT15- | ATAAATGCCT | | ATCaGAG | | TATCT | |
| Design 3 | C | | | | CT | |

| | | SEQ |
|---|---|---|
| | | ID |
| Nicking sgRNA name | protospacer | NO |
| 3307G > A NGA nsgRNA 1 | TATGTGGGAGACAGAGCTAT | 32 |
| 3307G > A NGA nsgRNA 2 | TGACCAGCACAATTGCTACT | 33 |
| 3307G > A NGA nsgRNA 3 | GGTTTCATTAATAAACCTCA | 34 |
| 3307G > A NGA nsgRNA 4 | ACTCCAACCCCTGTTTGCAT | 35 |

Prime Editing Analysis. The top ten most prevalent CRB1 mutations described in the LOVD database were evaluated for correction using prime editing. Only the use of both the canonical NGG PAM prime editor and the NGA PAM prime editor were considered. For those mutations with multiple possible designs, the NGG or NGA design that led to a nick closest to the mutation was chosen. Designs in the top DNA strand are labeled as positive (+) and those in the bottom strand are labeled as negative (−). Designs up to 10 nucleotides from the nick were included in the evaluation. Designs were made using only pegRNA; no nicking sgRNA designs were considered. Specific protospacers were identified for each mutation from which 3′-extensions were designed. Prime editing efficiencies are highly associated with the composition of the pegRNAs, as such optimizations of primer binding sequences (PBS) and reverse transcriptase (RT) template are necessary to achieve the best prime editing activities. Here, a PBS of 13 nucleotides in length and a RT template of 16 nucleotides in length were chosen, not accounting for insertions and deletions that require a longer RT.

### Example 1

Thirty combinations of prime editing guide RNA (pegRNA) and nicking single guide RNA (sgRNA) were tested per mutation, c.2843G>A (p.(Cys948Tyr)) and c.3307G>A (p.(Gly1103Arg)), on the corresponding CRB1 patient iPSC lines using nucleofection or Stem Cell lipofectamine. DNA was extracted 3 days after transfection/nucleofection. The target loci were amplified by PCR and editing efficiency was evaluated by Next Generation Sequencing (NGS) or Sanger sequencing followed by ICE analysis.

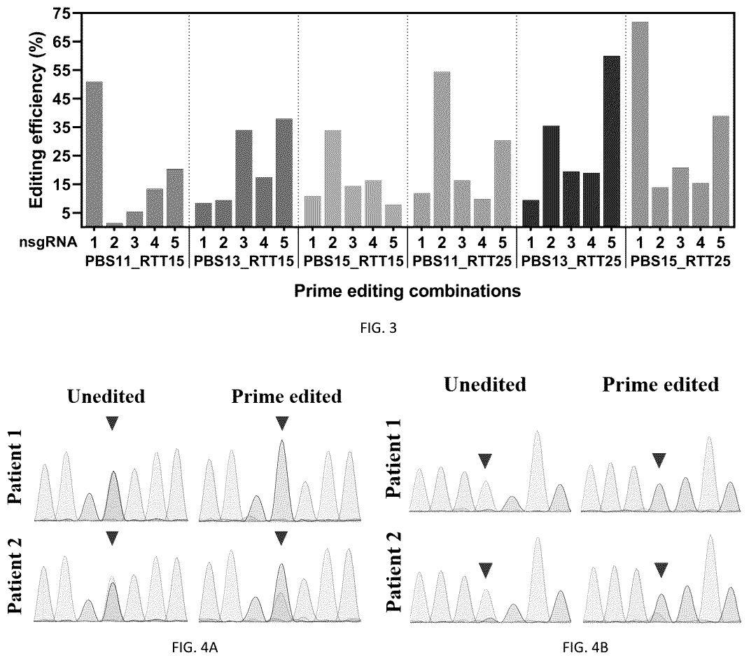

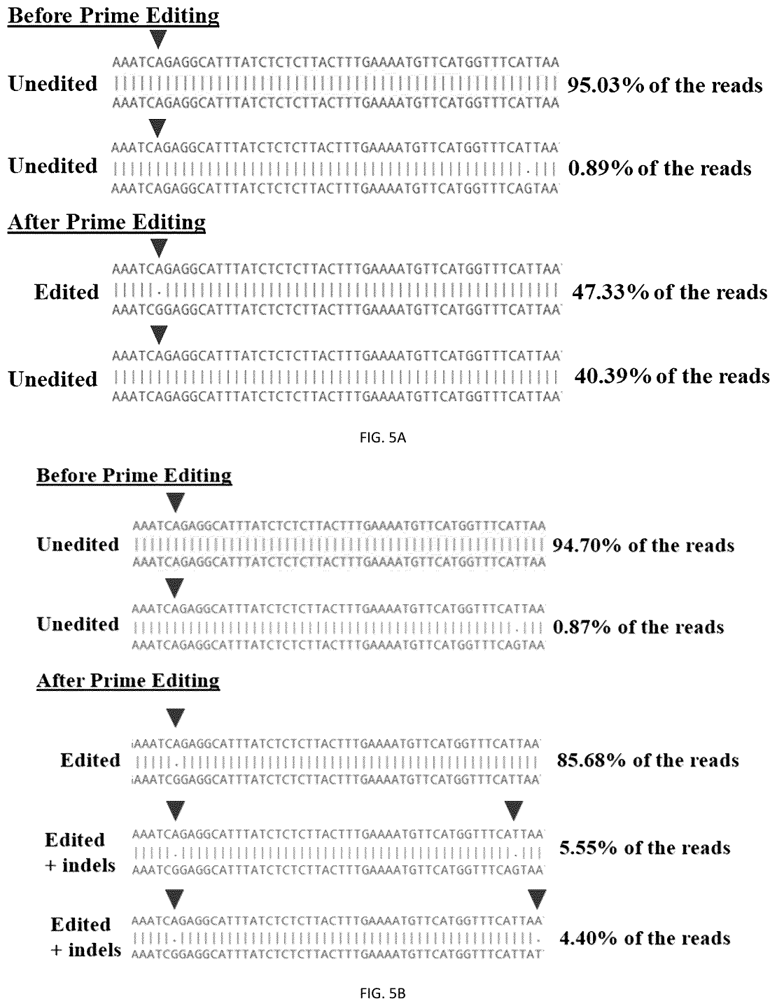

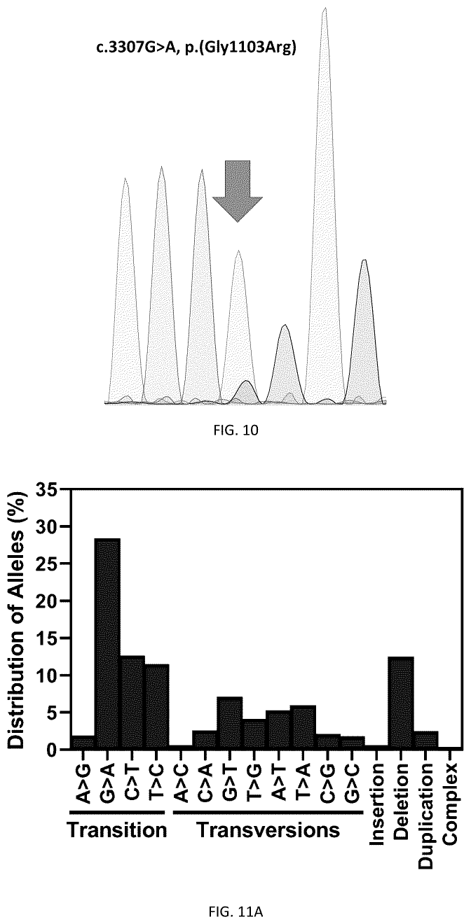

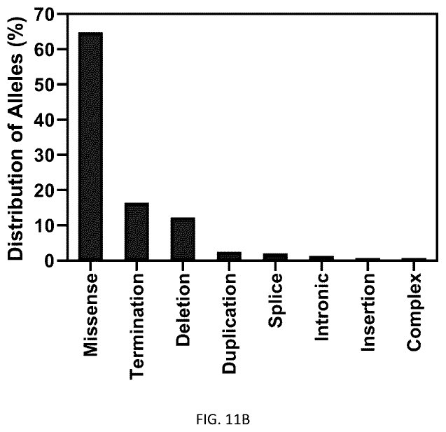

Contrary to conventional editors and base editing techniques, p.(Gly1103Arg) and p. (Cys948Tyr) patients' iPSC lines were amenable to prime editing, with editing efficiencies as high as 72% dependent on the combination of pegRNA (primer binding site (PBS) and reverse transcription template (RTT) length) and nicking sgRNA for a particular mutation. See FIGS. 3, 4, 5, and 7. As shown in FIG. 10, a split intein system may be utilized to deliver prime editing components to facilitate successful editing.

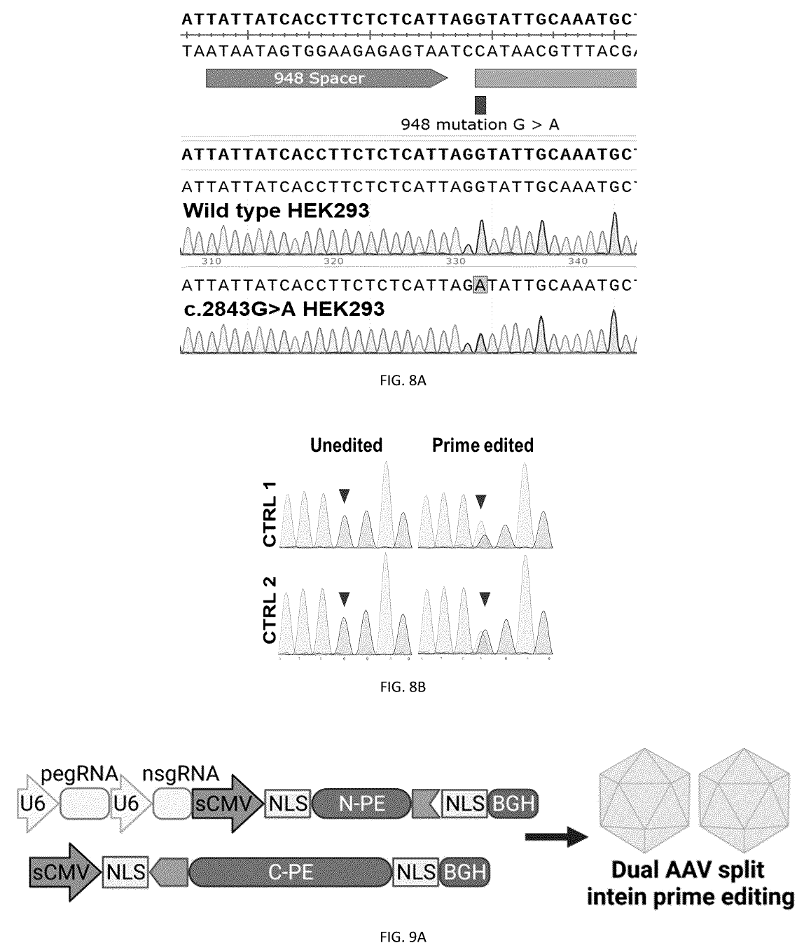

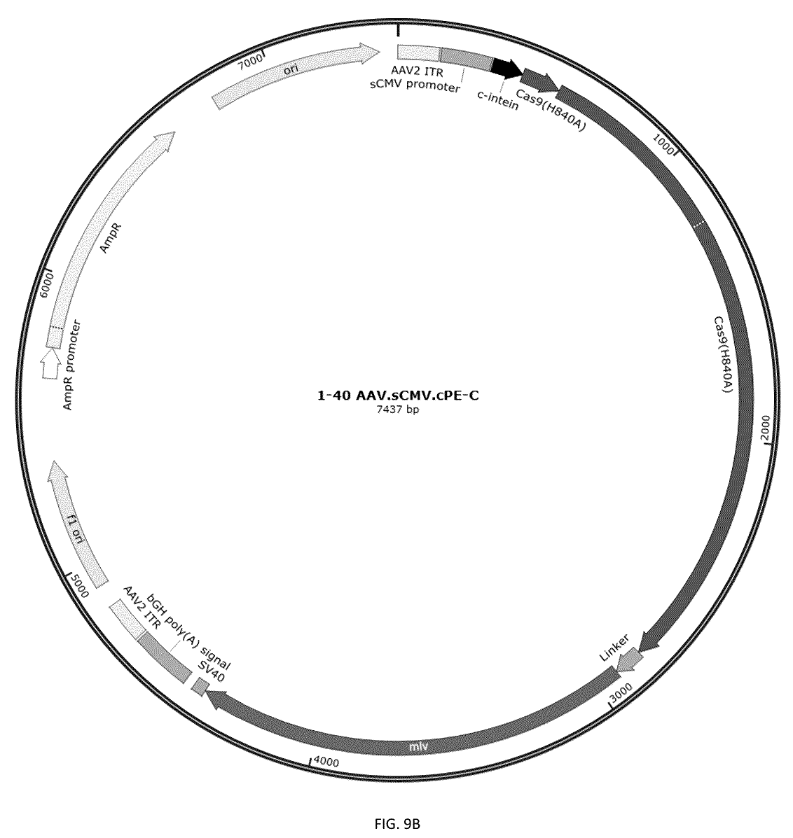

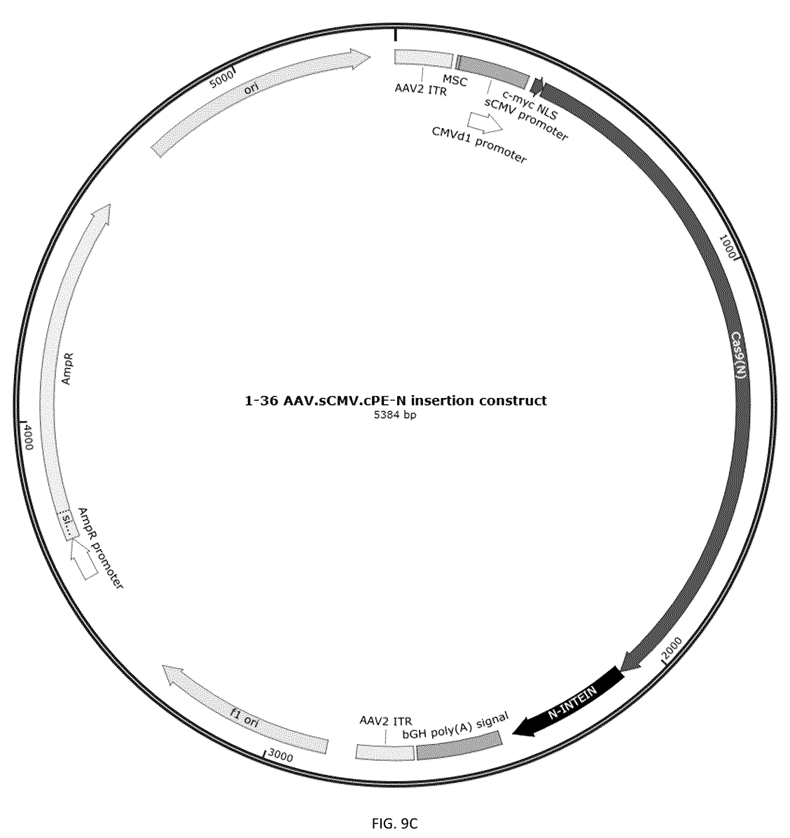

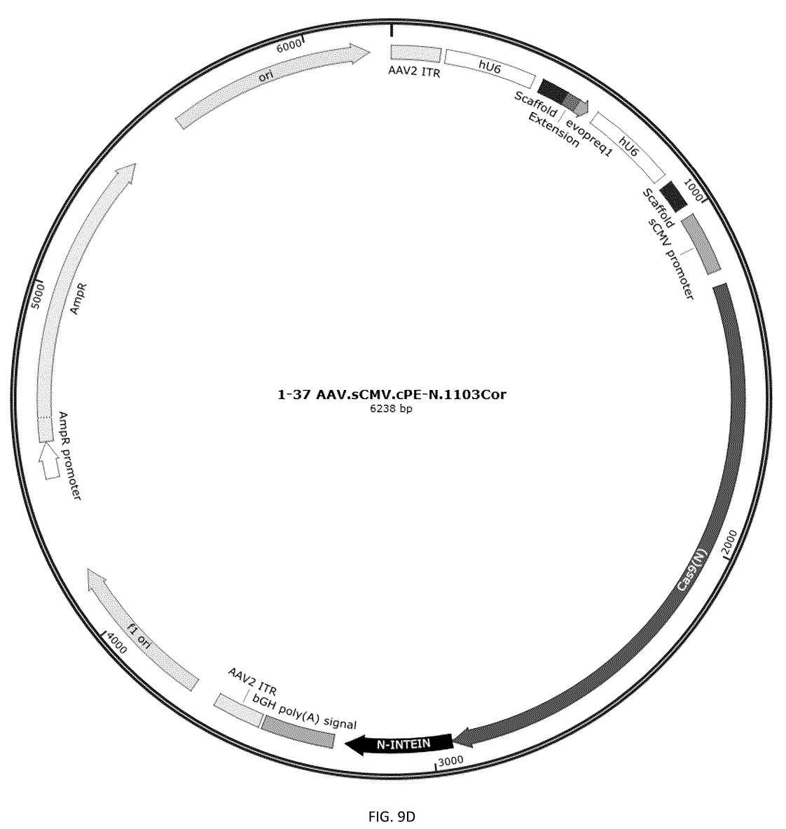

Prime editing was also used to insert the c.2843G>A (p.(Cys948Tyr)) and c.3307G>A (p.(Gly1103Arg)) mutations into control iPSCs and HEK293 cells (FIGS. 8A-8B).

### Example 2

To assess the prevalence and spectrum of CRB1 associated pathogenic variants amenable to base and prime editing, analysis of the Leiden Open Variation Database was carried out. Editable variants accounted for 54.5% for base editing and 99.8% for prime editing of all CRB1 pathogenic variants in the Leiden Open Variation Database. The 10 most common editable pathogenic variants for CRB1 accounted for 34.95% of all pathogenic variants, with the c.2843G>A, p.(Cys948Tyr) being the most common editable CRB1 variant.

Editable variants G>A (28.4%), C>T (12.6%), T>C (11.5%) and deletions (12.2%) were the most prevalent types (FIG. 11A) Missense variants were the predominant editable variants observed in the CRB1 gene (64.8%) followed by termination (define as SNVs that are associated with in-frame codons being altered to stop codons) (16.5%) and deletion variants (12.2%) (FIG. 11B).

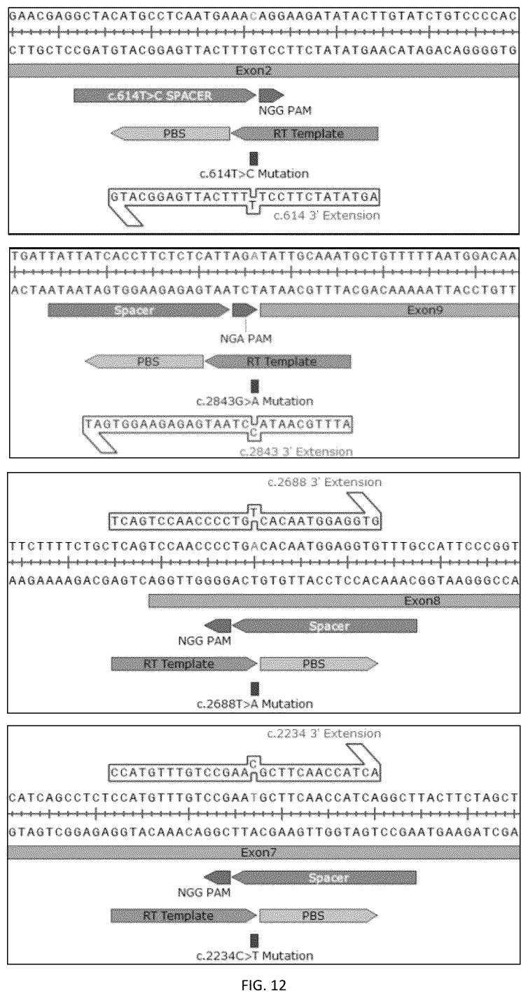

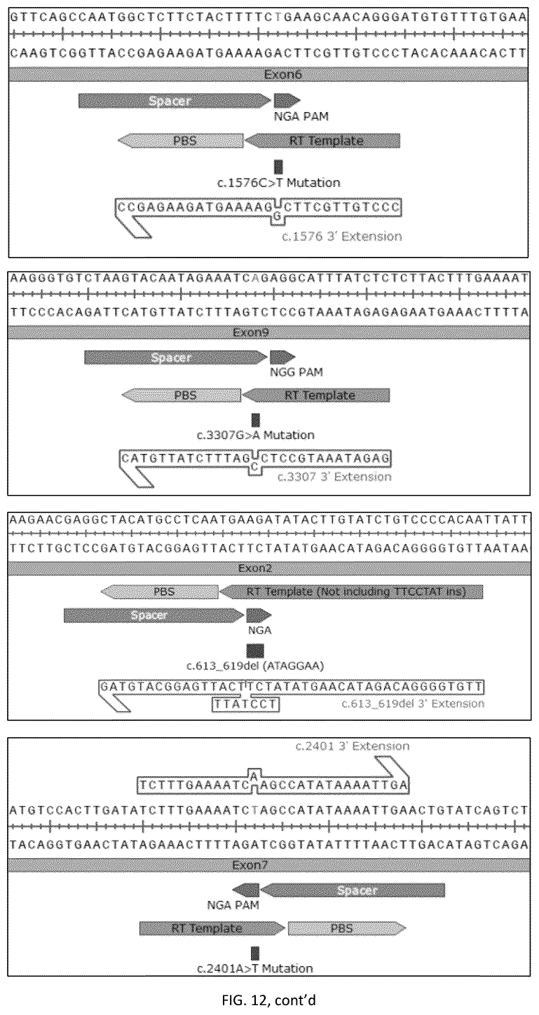

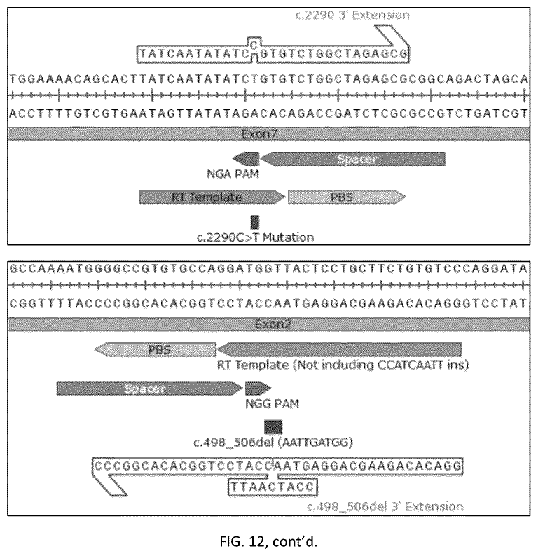

Of the top ten (RBI mutations, 6 are transitions, 2 are transversions, and 2 are deletions. All of those have suitable prime editing designs using either NGG or NGA PAM designs (Table 1). Mutations positioned far away from the PAM (at position ±33, for example) are still targetable by prime editing and can give rise to efficient editing as prime editing has been shown to have a low dependency on PAM placement. Here, example prime editing strategies with the edit positions close to the PAM are highlighted. All the top 10 CRB1 mutations show the ability to be prime editable (See FIG. 12 for a schematics of exemplary prime editing designs).

TABLE 1

| | | Edit | | SEQ ID | | SEQ ID |
|---|---|---|---|---|---|---|
| Mutation | PAM | position | Spacer | NO | 3′ Extension | NO |
| c.2843G > A | AGA | +6 | TATTATCA | 1 | ATTTGCAATACCTAATG | 44 |
| | | | CCTTCTCT | | AGAGAAGGTGAT | |
| | | | CATT | | | |
| c.2234C > T | CGG | −1 | AGCCTGAT | 36 | CCATGTTTGTCCGAACG | 45 |
| | | | GGTTGAAG | | CTTCAACCATCA | |
| | | | CATT | | | |
| c.2401A > T | AGA | −4 | CAGTTCAA | 37 | TCTTTGAAAATCAAGCC | 46 |
| | | | TTTTATAT | | ATATAAAATTGA | |
| | | | GGCT | | | |
| c.2290C > T | AGA | −4 | GCCGCGCT | 38 | TATCAATATATCCGTGTC | 47 |
| | | | CTAGCCAG | | TGGCTAGAGCG | |
| | | | ACAC | | | |
| c.2688T > A | GGG | −1 | CAAACACC | 39 | TCAGTCCAACCCCTGTC | 48 |
| | | | TCCATTGT | | ACAATGGAGGTG | |
| | | | GACA | | | |
| c.613_619del | AGA | +4 | GAGGCTAC | 40 | TTGTGGGGACAGATACA | 49 |
| | | | ATGCCTCA | | AGTATATCTTCCTATTTC | |
| | | | ATGA | | ATTGAGGCATGTAG | |
| c.498_506del | TGG | +6 | AATGGGG | 41 | GGACACAGAAGCAGGA | 50 |
| | | | CCGTGTGC | | GTAACCATCAATTCCAT | |
| | | | CAGGA | | CCTGGCACACGGCCC | |
| c.1576C > T | TGA | +4 | CAATGGCT | 42 | CCCTGTTGCTTCGGAAA | 51 |
| | | | CTTCTACT | | AGTAGAAGAGCC | |
| | | | TTTC | | | |
| c.3307G > A | AGG | +2 | CTAAGTAC | 17 | GAGATAAATGCCTCCGA | 52 |
| | | | AATAGAA | | TTTCTATTGTAC | |
| | | | ATCAG | | | |
| c.614T > C | AGG | +3 | GCTACATG | 43 | AGTATATCTTCCTTTTTC | 53 |
| | | | CCTCAATG | | ATTGAGGCATG | |
| | | | AAAC | | | |

### Example 3

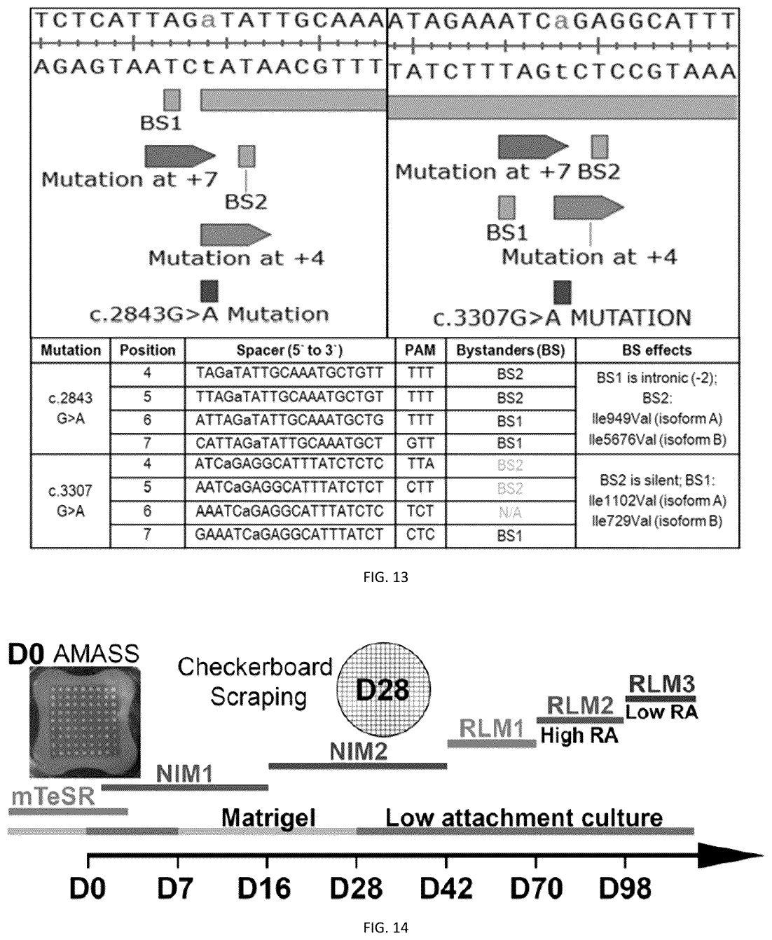

The disclosed methods tested the efficacy of prime editing for the correction of CRB1 mutations in patient induced pluripotent stem cell (iPSC) lines and developed the tools to test post mitotic editing using iPSCs-derived retinal organoids. Here prime editing was chosen over base editing (BE) to avoid bystanders, which facilitates the correction of some CRB1 mutations, including the c.2843G>A, p.Cys948Tyr, the most prevalent CRB1 mutation (FIGS. 13 and 14). Thirty combinations of prime editing guide RNA (pegRNA) and nicking single guide RNA (nsgRNA) design were tested per mutation on the corresponding CRB1 patient iPSC lines using nucleofection. Editing efficiency was evaluated by Next Generation Sequencing (NGS) or Sanger sequencing followed by ICE analysis. hiPSC-derived retinal organoids were generated as previously described (Tso, A et al., Methods Mol. Biol. 2023). An all-in-one prime editing and a split-intein prime editing strategies were developed for future evaluation of post mitotic editing using iPSCs-derived retinal organoids. Adeno-associated virus (AAV) and lipofectamine delivery methods were explored.

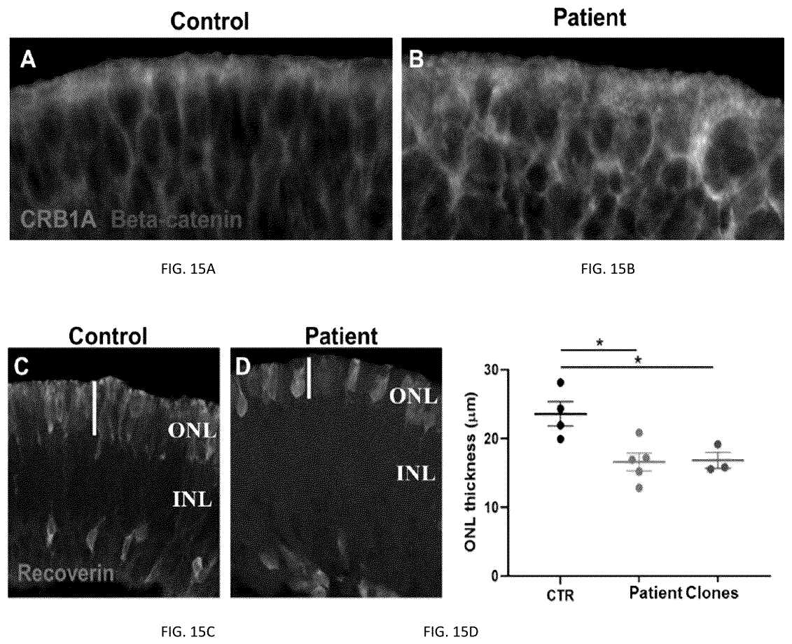

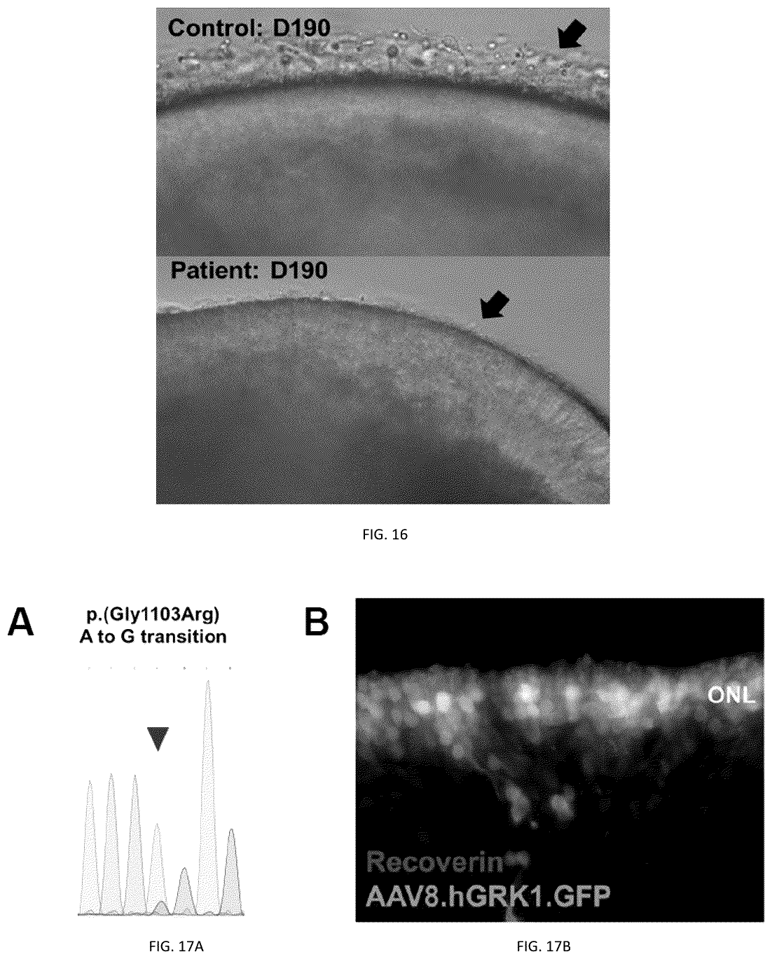

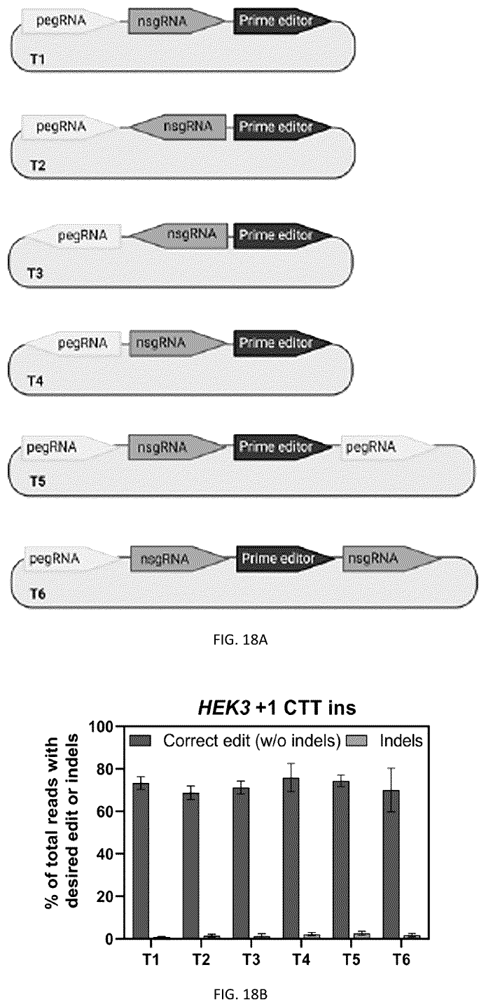

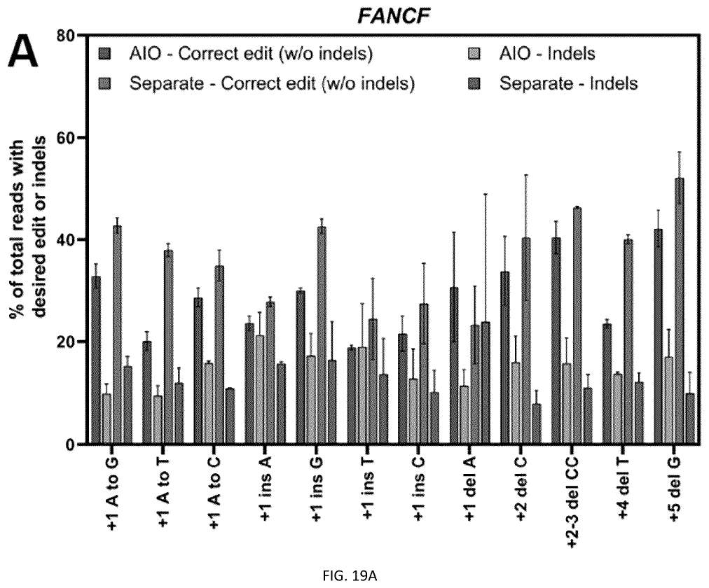

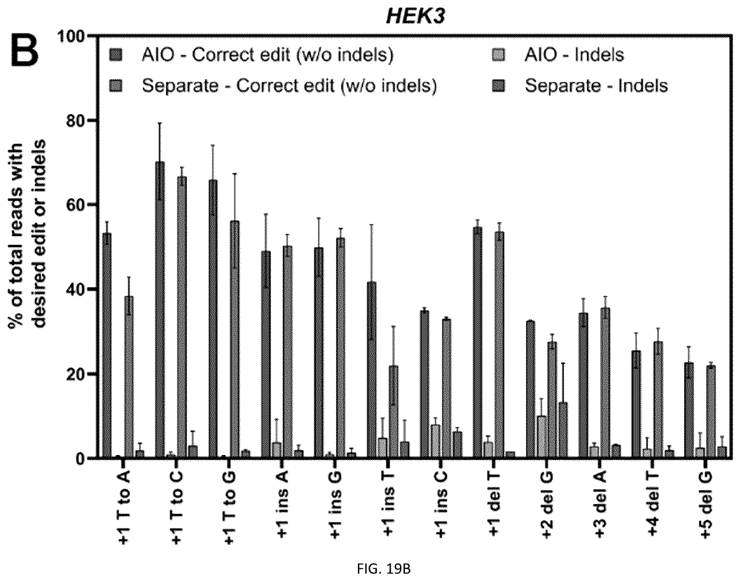

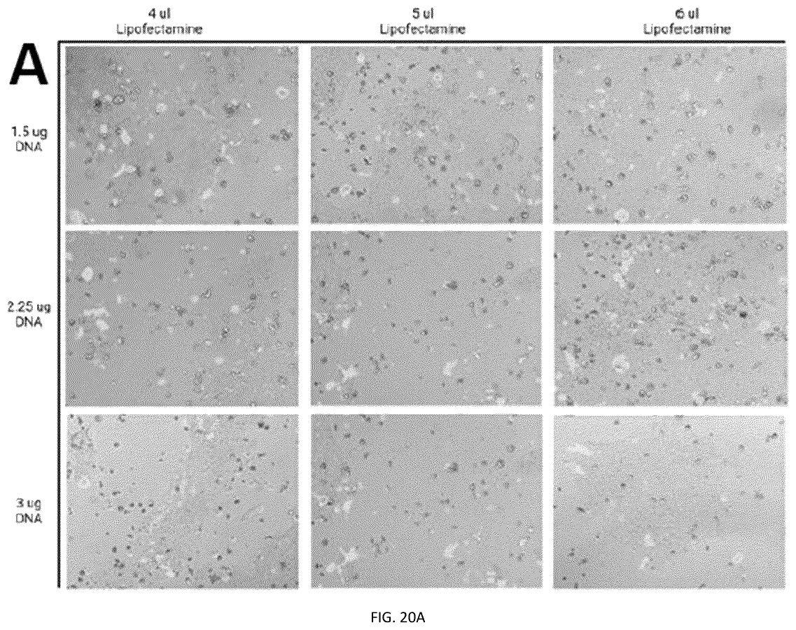

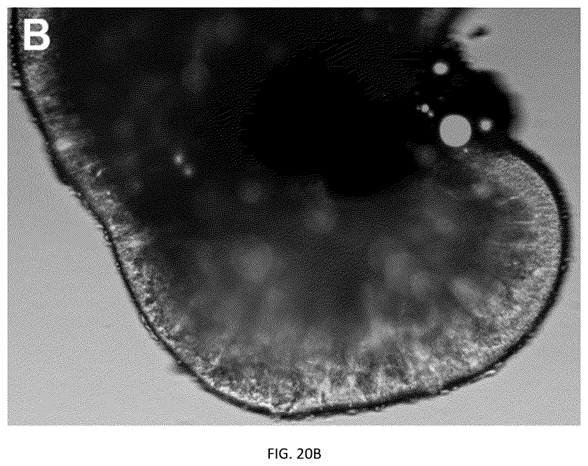

As described above, Prime editing was amenable for the correction of c.2843G>A CRB1 mutation (FIGS. 7 and 4A) and the c.3307G>A CRB1 mutation (FIGS. 3 and 4B). The split-intein system was able to correct the c.3307G>A CRB1 mutation (FIG. 17). CRB1 patient iPSCs-derived retinal organoids have clear phenotype (FIGS. 15 and 16). An all-in-one prime editing system was developed and validated for non-viral delivery (FIGS. 18-20).

As shown herein prime editing is amenable for the correction of the p.(Cys948Tyr) and p.(Gly1103Arg) CRB1 mutations. CRB1 patient derived retinal organoids have clear and reproducible phenotype's that serve as outcome measures for testing therapeutic efficacy. Split-intein prime editing strategy is viable for the correction of CRB1 mutations. Prime editing all-in-one system could be a valuable tool for creating isogenic controls and assessing post-mitotic editing in iPSC-derived retinal organoids.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A system for modifying the crumbs homologue-1 (CRB1) gene comprising:
a Cas protein, or a nucleic acid encoding thereof;
a reverse transcriptase, or a nucleic acid encoding thereof;
one or more RNA polynucleotides comprising a spacer sequence and an extension sequence comprising a primer binding sequence (PBS) and a reverse transcriptase template (RTT) sequence; or one or more nucleic acids encoding thereof; and
optionally, a nicking guide RNA (ngRNA), or a nucleic acid encoding thereof,
wherein the RTT sequence encodes one or more base substitutions, deletions, or additions to modify the CRB1 gene sequence.

2. The system of claim 1, wherein the spacer sequence and the extension sequence are contained within a single RNA polynucleotide.

3. The system of claim 1, wherein the spacer sequence comprises any of SEQ ID NOs: 1, 17, 23-25, and 36-43, the extension sequence comprises any of SEQ ID NOs: 44-56, the PBS comprises any of SEQ ID NOs: 4-6, 20-22, 30-31, the RTT sequence comprises any of SEQ ID NOs: 2-3, 12-13, 14, 18-19, 28-29, and 54-57, and the ngRNA comprises any of SEQ ID NOs: 7-11, 15-16, 23-27, and 32-35.

4. The system of claim 1, wherein the Cas protein is Cas9 or a variant or fragment thereof.

5. The system of claim 1, wherein the Cas protein is a Cas9 nickase.

6. The system of claim 1, wherein the Cas protein comprises a Cas protein variant configured to target an expanded range of PAM sequences.

7. The system of claim 1, wherein the Cas protein and the reverse transcriptase are contained within a single fusion protein.

8. The system of claim 1, wherein the CRB1 gene is a mutant CRB1 gene comprising one or more disease-causing mutations and wherein the RTT sequence corrects at least one disease-causing mutation in the CRB1 gene or wherein the CRB1 gene is a wild-type CRB1 gene and wherein the RTT sequence inserts at least one mutation in the CRB1 gene.

9. A method for modifying the crumbs homologue-1 (CRB1) gene comprising contacting a DNA encoding the CRB1 gene with a system of claim 1.

10. The method of claim 9, wherein the DNA encoding the CRB1 gene is in a cell and the contacting comprises introducing the system into the cell.

11. The method of claim 9, wherein the cell is in vitro, ex vivo, or in vivo.

12. The method of claim 10, wherein the cell is a human cell.

13. A method of treating or preventing a disease or disorder in a subject in need thereof comprising administering of a system of claim 1 to the subject,
wherein the disease or disorder is caused or mitigated by mutations in CRB1 gene.

14. The method of claim 13, wherein the disease or disorder comprises autosomal recessive retinitis pigmentosa (RP) or Leber congenital amaurosis (LCA).

15. The method of claim 13, wherein the system is configured for delivery to photoreceptor cells and/or Müller glial cells.


SEQUENCE LISTING (86 sequences)

SEQ ID NO: 1 - DNA, 20 bp, synthetic construct
  source 1..20: mol_type = other DNA
tattatcaccttctctcatt

SEQ ID NO: 2 - DNA, 16 bp, synthetic construct
  source 1..16: mol_type = other DNA
atttgcaatacctaat

SEQ ID NO: 3 - DNA, 26 bp, synthetic construct
  source 1..26: mol_type = other DNA
taaaaacagcatttgcaatacctaat

SEQ ID NO: 4 - DNA, 11 bp, synthetic construct
  source 1..11: mol_type = other DNA
gagagaaggtg

SEQ ID NO: 5 - DNA, 13 bp, synthetic construct
  source 1..13: mol_type = other DNA
gagagaaggtgat

SEQ ID NO: 6 - DNA, 15 bp, synthetic construct
  source 1..15: mol_type = other DNA
gagagaaggtgataa

SEQ ID NO: 7 - DNA, 20 bp, synthetic construct
  source 1..20: mol_type = other DNA
cattgcttctgaataatatt

SEQ ID NO: 8 - DNA, 20 bp, synthetic construct
  source 1..20: mol_type = other DNA
ggtaatattcccattgcttc

SEQ ID NO: 9 - DNA, 20 bp, synthetic construct
  source 1..20: mol_type = other DNA
gaaaccaaatgtgatattgg

SEQ ID NO: 10 - DNA, 20 bp, synthetic construct
  source 1..20: mol_type = other DNA
ccttgttctgaaaccaaatg

SEQ ID NO: 11 - DNA, 20 bp, synthetic construct
  source 1..20: mol_type = other DNA
attaccgttattaatagtaa

SEQ ID NO: 12 - DNA, 16 bp, synthetic construct
  source 1..16: mol_type = other DNA
atttgcgatgcctaat

SEQ ID NO: 13 - DNA, 26 bp, synthetic construct
  source 1..26: mol_type = other DNA
taaaaacagcatttgcgatgcctaat

SEQ ID NO: 14 - DNA, 26 bp, synthetic construct
  source 1..26: mol_type = other DNA
taaaaacagcatttgcaatatctaat

SEQ ID NO: 15 - DNA, 21 bp, synthetic construct
  source 1..21: mol_type = other DNA
gtgtgatattggtgagttctc

SEQ ID NO: 16 - DNA, 21 bp, synthetic construct
  source 1..21: mol_type = other DNA
gtctgaaaccaaatgtgatat

SEQ ID NO: 17 - DNA, 20 bp, synthetic construct
  source 1..20: mol_type = other DNA
ctaagtacaatagaaatcag

SEQ ID NO: 18 - DNA, 15 bp, synthetic construct
  source 1..15: mol_type = other DNA
agataaatgcctccg

SEQ ID NO: 19 - DNA, 25 bp, synthetic construct
  source 1..25: mol_type = other DNA
aaagtaagagagataaatgcctccg

SEQ ID NO: 20 - DNA, 11 bp, synthetic construct
  source 1..11: mol_type = other DNA
atttctattgt

SEQ ID NO: 21 - DNA, 13 bp, synthetic construct
  source 1..13: mol_type = other DNA
atttctattgtac

SEQ ID NO: 22 - DNA, 15 bp, synthetic construct
  source 1..15: mol_type = other DNA
atttctattgtactt

SEQ ID NO: 23 - DNA, 20 bp, synthetic construct
  source 1..20: mol_type = other DNA
ttgagaaattgctcttcctg

SEQ ID NO: 24 - DNA, 20 bp, synthetic construct
  source 1..20: mol_type = other DNA
acagccagtgaccactgaat

SEQ ID NO: 25 - DNA, 20 bp, synthetic construct
  source 1..20: mol_type = other DNA
tcctccatgcaaacaggggt

SEQ ID NO: 26 - DNA, 20 bp, synthetic construct
  source 1..20: mol_type = other DNA
tattatccttcaaaaagttg

SEQ ID NO: 27 - DNA, 20 bp, synthetic construct
  source 1..20: mol_type = other DNA
ttgtacttagacacccttgc

SEQ ID NO: 28 - DNA, 15 bp, synthetic construct
  source 1..15: mol_type = other DNA
agataaattccgcca

SEQ ID NO: 29 - DNA, 25 bp, synthetic construct
  source 1..25: mol_type = other DNA
aaagtaagagagataaattccgcca

SEQ ID NO: 30 - DNA, 12 bp, synthetic construct
  source 1..12: mol_type = other DNA
tctattgtactt

SEQ ID NO: 31 - DNA, 12 bp, synthetic construct
  source 1..12: mol_type = other DNA
gcatttatctct

SEQ ID NO: 32 - DNA, 20 bp, synthetic construct
  source 1..20: mol_type = other DNA
tatgtgggagacagagctat

SEQ ID NO: 33 - DNA, 20 bp, synthetic construct
  source 1..20: mol_type = other DNA
tgaccagcacaattgctact

SEQ ID NO: 34 - DNA, 20 bp, synthetic construct
  source 1..20: mol_type = other DNA
ggtttcattaataaacctca

SEQ ID NO: 35 - DNA, 20 bp, synthetic construct
  source 1..20: mol_type = other DNA
actccaacccctgtttgcat

SEQ ID NO: 36 - DNA, 20 bp, synthetic construct
  source 1..20: mol_type = other DNA
agcctgatggttgaagcatt

SEQ ID NO: 37 - DNA, 20 bp, synthetic construct
  source 1..20: mol_type = other DNA
cagttcaattttatatggct

SEQ ID NO: 38 - DNA, 20 bp, synthetic construct
  source 1..20: mol_type = other DNA
gccgcgctctagccagacac

SEQ ID NO: 39 - DNA, 20 bp, synthetic construct
  source 1..20: mol_type = other DNA
caaacacctccattgtgaca

SEQ ID NO: 40 - DNA, 20 bp, synthetic construct
  source 1..20: mol_type = other DNA
gaggctacatgcctcaatga

SEQ ID NO: 41 - DNA, 20 bp, synthetic construct
  source 1..20: mol_type = other DNA
aatggggccgtgtgccagga

SEQ ID NO: 42 - DNA, 20 bp, synthetic construct
  source 1..20: mol_type = other DNA
caatggctcttctacttttc

SEQ ID NO: 43 - DNA, 20 bp, synthetic construct
  source 1..20: mol_type = other DNA
gctacatgcctcaatgaaac

SEQ ID NO: 44 - DNA, 29 bp, synthetic construct
  source 1..29: mol_type = other DNA
atttgcaatacctaatgagagaaggtgat

SEQ ID NO: 45 - DNA, 29 bp, synthetic construct
  source 1..29: mol_type = other DNA
ccatgtttgtccgaacgcttcaaccatca

SEQ ID NO: 46 - DNA, 29 bp, synthetic construct
  source 1..29: mol_type = other DNA
tctttgaaaatcaagccatataaaattga

SEQ ID NO: 47 - DNA, 29 bp, synthetic construct
  source 1..29: mol_type = other DNA
tatcaatatatccgtgtctggctagagcg

SEQ ID NO: 48 - DNA, 29 bp, synthetic construct
  source 1..29: mol_type = other DNA
tcagtccaacccctgtcacaatggaggtg

SEQ ID NO: 49 - DNA, 49 bp, synthetic construct
  source 1..49: mol_type = other DNA
ttgtggggacagatacaagtatatcttcctatttcattgaggcatgtag

SEQ ID NO: 50 - DNA, 48 bp, synthetic construct
  source 1..48: mol_type = other DNA
ggacacagaagcaggagtaaccatcaattccatcctggcacacggccc

SEQ ID NO: 51 - DNA, 29 bp, synthetic construct
  source 1..29: mol_type = other DNA
ccctgttgcttcggaaaagtagaagagcc

SEQ ID NO: 52 - DNA, 29 bp, synthetic construct
  source 1..29: mol_type = other DNA
gagataaatgcctccgatttctattgtac

SEQ ID NO: 53 - DNA, 29 bp, synthetic construct
  source 1..29: mol_type = other DNA
agtatatcttcctttttcattgaggcatg

SEQ ID NO: 54 - DNA, 25 bp, synthetic construct
  source 1..25: mol_type = other DNA
aaagtaagagagataaatgcctctg

SEQ ID NO: 55 - DNA, 15 bp, synthetic construct
  source 1..15: mol_type = other DNA
agataaatgcctctg

SEQ ID NO: 56 - DNA, 15 bp, synthetic construct
  source 1..15: mol_type = other DNA
taaatgcctctgatt

SEQ ID NO: 57 - DNA, 15 bp, synthetic construct
  source 1..15: mol_type = other DNA
caatagaaatcagag

SEQ ID NO: 58 - DNA, 52 bp, synthetic construct
  source 1..52: mol_type = other DNA
aaatcagaggcatttatctctcttactttgaaaatgttcatggtttcattaa

SEQ ID NO: 59 - DNA, 52 bp, synthetic construct
  source 1..52: mol_type = other DNA
aaatcagaggcatttatctctcttactttgaaaatgttcatggtttcagtaa

SEQ ID NO: 60 - DNA, 52 bp, synthetic construct
  source 1..52: mol_type = other DNA
aaatcggaggcatttatctctcttactttgaaaatgttcatggtttcattaa

SEQ ID NO: 61 - DNA, 52 bp, synthetic construct
  source 1..52: mol_type = other DNA
aaatcggaggcatttatctctcttactttgaaaatgttcatggtttcagtaa

SEQ ID NO: 62 - DNA, 52 bp, synthetic construct
  source 1..52: mol_type = other DNA
aaatcggaggcatttatctctcttactttgaaaatgttcatggtttcattat

SEQ ID NO: 63 - DNA, 37 bp, synthetic construct
  source 1..37: mol_type = other DNA
attattatcaccttctctcattaggtattgcaaatgc

SEQ ID NO: 64 - DNA, 37 bp, synthetic construct
  source 1..37: mol_type = other DNA
attattatcaccttctctcattagatattgcaaatgc

SEQ ID NO: 65 - DNA, 55 bp, synthetic construct
  source 1..55: mol_type = other DNA
gaacgaggctacatgcctcaatgaaacaggaagatatacttgtatctgtccccac

SEQ ID NO: 66 - DNA, 55 bp, synthetic construct
  source 1..55: mol_type = other DNA
tgattattatcaccttctctcattagatattgcaaatgctgtttttaatggacaa

SEQ ID NO: 67 - DNA, 55 bp, synthetic construct
  source 1..55: mol_type = other DNA
ttcttttctgctcagtccaacccctgacacaatggaggtgtttgccattcccggt

SEQ ID NO: 68 - DNA, 55 bp, synthetic construct
  source 1..55: mol_type = other DNA
catcagcctctccatgtttgtccgaatgcttcaaccatcaggcttacttctagct

SEQ ID NO: 69 - DNA, 53 bp, synthetic construct
  source 1..53: mol_type = other DNA
gttcagccaatggctcttctacttttctgaagcaacagggatgtgtttgtgaa

SEQ ID NO: 70 - DNA, 56 bp, synthetic construct
  source 1..56: mol_type = other DNA
aagggtgtctaagtacaatagaaatcagaggcatttatctctcttactttgaaaat

SEQ ID NO: 71 - DNA, 57 bp, synthetic construct
  source 1..57: mol_type = other DNA
aagaacgaggctacatgcctcaatgaagatatacttgtatctgtccccacaattatt

SEQ ID NO: 72 - DNA, 56 bp, synthetic construct
  source 1..56: mol_type = other DNA
atgtccacttgatatctttgaaaatctagccatataaaattgaactgtatcagtct

SEQ ID NO: 73 - DNA, 56 bp, synthetic construct
  source 1..56: mol_type = other DNA
tggaaaacagcacttatcaatatatctgtgtctggctagagcgcggcagactagca

SEQ ID NO: 74 - DNA, 55 bp, synthetic construct
  source 1..55: mol_type = other DNA
gccaaaatggggccgtgtgccaggatggttactcctgcttctgtgtcccaggata

SEQ ID NO: 75 - DNA, 19 bp, synthetic construct
  source 1..19: mol_type = other DNA
tctcattagatattgcaaa

SEQ ID NO: 76 - DNA, 19 bp, synthetic construct
  source 1..19: mol_type = other DNA
tttgcaatatctaatgaga

SEQ ID NO: 77 - DNA, 20 bp, synthetic construct
  source 1..20: mol_type = other DNA
tagatattgcaaatgctgtt

SEQ ID NO: 78 - DNA, 20 bp, synthetic construct
  source 1..20: mol_type = other DNA
ttagatattgcaaatgctgt

SEQ ID NO: 79 - DNA, 20 bp, synthetic construct
  source 1..20: mol_type = other DNA
attagatattgcaaatgctg

SEQ ID NO: 80 - DNA, 20 bp, synthetic construct
  source 1..20: mol_type = other DNA
cattagatattgcaaatgct

SEQ ID NO: 81 - DNA, 19 bp, synthetic construct
  source 1..19: mol_type = other DNA
atagaaatcagaggcattt

SEQ ID NO: 82 - DNA, 19 bp, synthetic construct
  source 1..19: mol_type = other DNA
aaatgcctctgatttctat

SEQ ID NO: 83 - DNA, 20 bp, synthetic construct
  source 1..20: mol_type = other DNA
atcagaggcatttatctctc

SEQ ID NO: 84 - DNA, 20 bp, synthetic construct
  source 1..20: mol_type = other DNA
aatcagaggcatttatctct

SEQ ID NO: 85 - DNA, 20 bp, synthetic construct
  source 1..20: mol_type = other DNA
aaatcagaggcatttatctc

SEQ ID NO: 86 - DNA, 20 bp, synthetic construct
  source 1..20: mol_type = other DNA
gaaatcagaggcatttatct